(19) United States
(12) Patent Application Publication
(10) Pub. No.: US 2025/0235220 A1
(43) Pub. Date: Jul. 24, 2025

(54) THROMBECTOMY APPARATUSES

(71) Applicant: Boston Scientific Scimed, Inc., Maple Grove, MN (US)

(72) Inventors: Michael Dotsey, Chester Springs, PA (US); Andrew Miller, Phoenixville, PA (US); Robert J. Garabedian, Newark, CA (US); Terrence Haas, Plymouth, MN (US)

(73) Assignee: Boston Scientific Scimed, Inc., Maple Grove, MN (US)

(21) Appl. No.: 18/985,929

(22) Filed: Dec. 18, 2024

Related U.S. Application Data
(60) Provisional application No. 63/611,899, filed on Dec. 19, 2023.

(51) Int. Cl. A61B 17/22; A61B 17/00; A61B 90/00

(52) U.S. Cl. CPC A61B 17/22; A61B 2017/00292; A61B 2017/00424; A61B 2017/0046; A61B 2090/064

(57) ABSTRACT

An assembly for engaging a medical device shaft includes an assembly body adapted to be held in a person's hand. The assembly body may be an ergonomic assembly body, for example. The assembly body defines a channel adapted to accommodate a medical device shaft extending therethrough. An engagement feature is adapted to selectively engage an engagement member secured on the medical device shaft, the engagement feature movable between an open configuration in which the engagement feature does not engage the engagement member and a closed configuration in which the engagement feature engages the engagement member.

### CROSS-REFERENCE TO RELATED APPLICATIONS

This application claims the benefit of priority under 35 U.S.C. § 119 of U.S. Provisional Application No. 63/611,899, filed Dec. 19, 2023, the entire disclosure of which is hereby incorporated by reference.

### TECHNICAL FIELD

The present disclosure pertains to medical devices, and methods for manufacturing and using medical devices. More particularly, the present disclosure pertains to medical devices, methods, and systems for performing thrombectomy procedures.

### BACKGROUND

Removal of material from within a vessel or chamber is often useful. For example, removal of tissue such as blood clots from within a vasculature may improve patient conditions and quality of life. Clot removal may be beneficial or even necessary to improve patient outcomes. For example, in the peripheral vasculature, interventions and procedures can reduce the need for an amputation by 80 percent. The ultimate goal of any technique to treat these conditions is to remove the blockage and to restore patency, quickly, safely, and cost effectively.

Devices for mechanically removing material, including thrombus material, from with a lumen of the vessel may include an inverting tube for removing material from a body lumen, such as for removing a clot from a blood vessel (e.g., thrombectomy devices), are disclosed and described in each of U.S. Pat. Nos. 10,271,864, 10,835,234, 10,561,431, 10,842,513 and 11,253,291. The described apparatuses may face challenges when removing large amounts of material which may require multiple attempts to remove, and/or when removing softer clot material, which may be difficult to grip. In some cases, it may be difficult to completely remove clot material from against the wall of the vessel. Thus, there is an ongoing need for devices, including thrombectomy devices, that can remove tissue, and particularly large and/or soft materials, from within a body lumen.

### SUMMARY

This disclosure provides design, material, manufacturing method, and use alternatives for medical devices. An example may be found in an assembly for engaging a medical device shaft. The assembly includes an assembly body adapted to be held in a person's hand. The assembly body defines a channel adapted to accommodate the medical device shaft extending therethrough. The assembly includes an engagement feature adapted to selectively engage an engagement member secured on the medical device shaft. The engagement feature is movable between an open configuration in which the engagement feature does not engage the engagement member and a closed configuration in which the engagement feature engages the engagement member.

Alternatively or additionally, the engagement feature may include a first engagement feature element molded into the assembly body. The first engagement feature element is biased into the open configuration. A second engagement feature may be slidingly disposed relative to the first engagement feature such that sliding the second engagement feature over the first engagement feature element moves the first engagement feature element into the closed configuration.

Alternatively or additionally, the assembly may further include a lever pivotably secured to the assembly body. The engagement feature may include a tab extending towards the channel. The tab extends into the channel when the engagement feature is in the closed configuration.

Alternatively or additionally, the assembly body may include a first assembly body half extending from an assembly body midpoint and a second assembly body half extending from the assembly body midpoint. The engagement feature may include a first engagement feature element disposed within the first assembly body half and a second engagement feature element disposed within the second assembly body half. The first assembly body half and the second assembly body half are biased to the open configuration in which the first engagement feature element and the second engagement feature element are spaced apart. The first assembly body half and the second assembly body half are adapted to be squeezed together in order to move the first engagement feature element and the second engagement feature element together into the closed configuration.

Alternatively or additionally, the engagement feature may include a spring-loaded collet disposed within the channel that is adapted to push against one or more ramp surfaces within the assembly body, and the spring-loaded collet may open to accommodate the engagement member as the engagement member passes, then closes to hold engagement member.

Alternatively or additionally, the engagement feature may further include a twist element that when rotated causes the spring-loaded collet to open.

Alternatively or additionally, the assembly may further include sliders that are disposed on either side of the assembly body. The sliders are operably coupled with the spring-loaded collet and are adapted to allow manually opening the spring-loaded collet by sliding the sliders.

Alternatively or additionally, the assembly body may include a first assembly body section and a second assembly body section that is hingedly coupled to the first assembly body section. The engagement feature may include a first engagement feature element disposed within the first assembly body section and a second engagement feature element disposed within the second assembly body section. The second housing section may be adapted to pivot away from the first assembly body section in the open configuration and to pivot towards the first assembly body section in the closed configuration.

Alternatively or additionally, the second assembly body section may be hingedly coupled to the first assembly body section via a living hinge.

Alternatively or additionally, the first engagement feature element and the second engagement feature element may each be disposed at an end of the assembly body opposite that of the living hinge.

Alternatively or additionally, the first engagement feature element and the second engagement feature element may each be disposed proximate the living hinge.

Alternatively or additionally, the engagement feature may include a first engagement feature element with a first jaw portion and a first handle portion and a second engagement feature element with a second jaw portion and a second handle portion. The first jaw portion may be biased into contact with the second jaw portion.

Alternatively or additionally, the first jaw portion may include a first ramp portion, the second jaw portion may include a second ramp portion, and the first ramp portion and the second ramp portion together may allow the engagement member to urge the first jaw portion and the second jaw portion away from their biased position such that the engagement member can be pushed into engagement with the engagement feature.

Alternatively or additionally, the assembly body may include a first side aperture and a second side aperture. The first handle portion may extend out the first side aperture, the second handle portion may extend out the second side aperture, and the first handle portion and the second handle portion may be adapted to be pushed in order to separate the first jaw portion and the second jaw portion.

Alternatively or additionally, the assembly may further include a force gauge.

Alternatively or additionally, the assembly body may include an ergonomic shape to facilitate being held in a person's hand.

Another example may be found in an assembly for engaging a medical device shaft. The assembly includes an assembly body adapted to be held in a person's hand. The assembly body defines a channel adapted to accommodate the medical device shaft extending therethrough. An engagement feature is adapted to engage an engagement member secured on the medical device shaft. A force gauge is secured relative to the assembly body and is adapted to provide an indication of an applied force.

Alternatively or additionally, the assembly body may include an ergonomic shape to facilitate being held in a person's hand.

Alternatively or additionally, the engagement feature may be adapted to releasably engage the engagement member.

Another example may be found in an assembly for engaging a medical device shaft that includes an engagement member. The assembly includes an ergonomic assembly body that is adapted to be held in a person's hand and defines a channel adapted to accommodate the medical device shaft extending therethrough. An engagement feature is adapted to selectively engage the engagement member and is movable between an open configuration in which the engagement feature does not engage the engagement member and a closed configuration in which the engagement feature engages the engagement member.

The above summary of some embodiments is not intended to describe each disclosed embodiment or every implementation of the present disclosure. The Figures and Detailed Description, which follow, more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1A shows an inverting tube (e.g., thrombectomy) apparatus that may be used to remove material from a vessel, shown in a side view with an inversion support catheter and a flexible outer tube;

FIG. 1B shows an inverting tube (e.g., thrombectomy) apparatus that may be used to remove material from a vessel, shown in a vessel, proximal to a clot;

FIG. 1C illustrates the removal of a clot from the vessel using the apparatus of FIG. 1A, by pulling the flexible tube on the outside of the inversion support catheter proximally so that it rolls over the distal end of the inversion support catheter and into the inversion support catheter, drawing the clot with it;

FIG. 2A shows an inverting tube apparatus including an expandable funnel that is attached to a distal end of an inversion support catheter;

FIG. 2B shows the inverting tube apparatus of FIG. 2A in a deployed configuration with an intermediate (e.g., delivery) catheter withdrawn proximally so that the expandable funnel at the distal end of the inversion support catheter can expand;

FIG. 3A is a top plan view of an illustrative assembly shown in combination with a puller extending through the assembly in an open configuration;

FIG. 3B is a perspective view of the assembly of FIG. 3A in an open configuration;

FIG. 3C is a perspective view of the assembly of FIG. 3A showing the assembly within a user's hand and in combination with a puller, shown in a closed configuration;

FIG. 4A is a perspective view of an illustrative assembly shown in an open configuration;

FIG. 4B is a side view of the illustrative assembly of FIG. 4A;

FIG. 5A is a perspective view of an illustrative assembly, shown in an open configuration;

FIG. 5B is a side view of the illustrative assembly of FIG. 5A, shown in an open configuration;

FIG. 5C is a side view of the illustrative assembly of FIG. 5A, shown in a closed configuration;

FIG. 6A is a perspective view of an illustrative assembly, shown in combination with a puller and in a closed configuration;

FIG. 6B is a side view of the illustrative assembly of FIG. 6A, shown in an open configuration;

FIG. 7A is a perspective view of an illustrative assembly, shown in a closed configuration;

FIG. 7B is a perspective view of the illustrative assembly of FIG. 7A, shown in an open configuration;

FIG. 8A is a side view of an illustrative assembly, shown in combination with a puller and in an open configuration;

FIG. 8B is a side view of the illustrative assembly of FIG. 8A, shown in combination with a puller and in a closed configuration;

FIG. 9A is a perspective view of an illustrative assembly, shown in a closed configuration;

FIG. 9B is a cross-sectional view taken along the line 9B-9B in FIG. 9A;

FIG. 10A is a perspective view of an illustrative assembly, shown in a closed configuration;

FIG. 10B is a side view of a portion of the illustrative assembly of FIG. 10A, shown in an open configuration;

FIG. 10C is a side view of a portion of the illustrative assembly of FIG. 10A, shown in a closed configuration;

FIG. 11A is a perspective view of an illustrative assembly, shown in a closed configuration;

FIG. 11B is a cross-sectional view taken along the line 11B-11B of FIG. 11A;

FIG. 11C is a perspective view of a first illustrative engagement feature element forming part of the illustrative assembly of FIG. 11A;

FIG. 11D is a perspective view of a second illustrative engagement feature element forming part of the illustrative assembly of FIG. 11A;

FIG. 12A is a perspective view of an illustrative assembly, shown in a closed configuration;

FIG. 12B is a cross-sectional view taken along the line 12B-12B of FIG. 12A;

FIG. 12C is a perspective view of an engagement feature forming part of the illustrative assembly of FIG. 12A;

FIG. 13A is a perspective view of an illustrative assembly;

FIG. 13B is a perspective view of an illustrative engagement feature forming part of the illustrative assembly of FIG. 13A;

FIG. 14 is a perspective view of an illustrative assembly that includes force feedback;

FIG. 15A is a perspective view of an illustrative assembly, shown in combination with a puller;

FIG. 15B is a cross-sectional view taken along the line 15B-15B of FIG. 15A;

FIG. 16A is a perspective view of an illustrative assembly;

FIG. 16B is a bottom plan view of the illustrative assembly of FIG. 16A;

FIG. 17A is a perspective view of an illustrative assembly;

FIG. 17B is a bottom plan view of the illustrative assembly of FIG. 17A;

FIG. 18 is a perspective view of an illustrative assembly kit that is customizable for differing puller sizes;

FIG. 19 is a perspective view of an illustrative assembly including an insert that is customizable for differing puller sizes;

FIG. 20A is a perspective view of an illustrative assembly, shown in combination with a puller;

FIG. 20B is a cross-sectional view taken along the line 20B-20B of FIG. 21;

FIG. 21A is a front perspective view of an illustrative assembly, shown in the grasp of a user's hand;

FIG. 21B is a rear perspective view of the illustrative assembly of FIG. 21A, shown in the grasp of a user's hand;

FIG. 22A is a perspective view of an illustrative assembly, shown in the grasp of a user's hand in a first orientation;

FIG. 22B is a perspective view of the illustrative assembly of FIG. 22A, shown in the grasp of a user's hand in a second orientation;

FIG. 23 is a perspective view of an illustrative assembly, shown in the grasp of a user's hand; and

FIG. 24 is a perspective view of an illustrative assembly, shown in the grasp of a user's hand.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

The methods and apparatuses described herein may also relate to improvement in the operation, and in particular, the insertion and use of, inverting tube apparatuses for removing material from within a body. These apparatuses may generally include an inversion support, which may include a catheter and in some examples a funnel region at the distal end of the catheter, a flexible tube configured to move over the outside of the inversion support and invert into the inversion support, and in some examples a puller attached to a first end of the inversion support for pulling the flexible tube into the inversion support. These apparatuses may be generally referred to as apparatuses for removing a material from a vessel and may be configured as mechanical thrombectomy apparatuses.

Also described herein are systems and methods for improving the ability of the flexible tube to grab and extract material from the walls of the body lumen. Also described herein are methods for enhancing or improving the ability of the apparatus to grab and draw in a clot by creating slack into the flexible tube before it inverts over the distal end of the inversion support catheter.

Any of these features, components and techniques may be used separately or in combination.

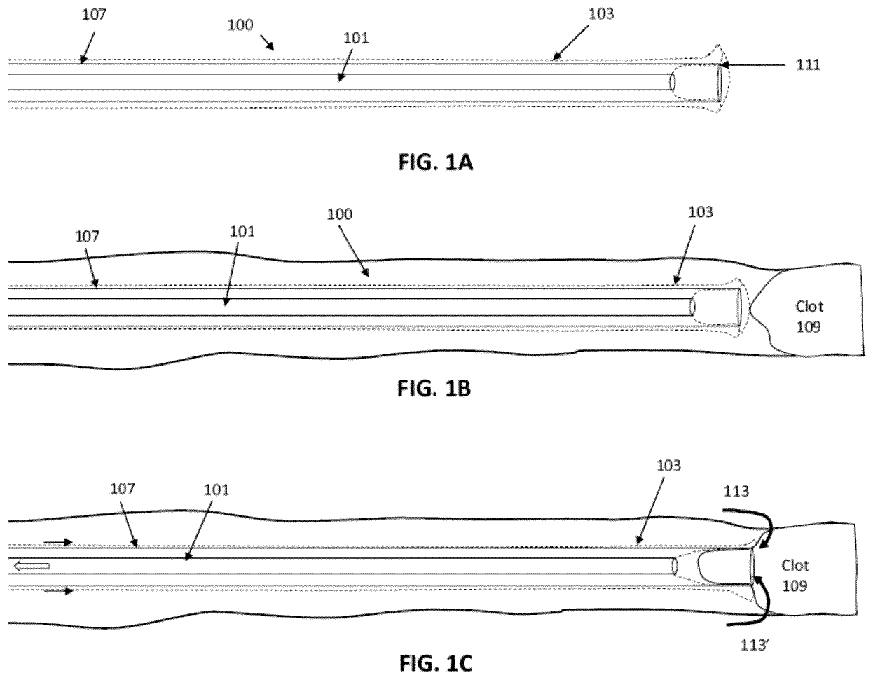

In general, an inverting tube apparatus (also referred to herein as “mechanical thrombectomy apparatus” or “inverting thrombectomy apparatus”) may be configured to remove material, such as a clot, using a length of inverting tube, as shown in FIGS. 1A-1C. The apparatuses and methods of using them described herein may be used within the vasculature, including the neuro-vasculature and the peripheral vasculature.

For example, FIG. 1A provides an example of an inverting thrombectomy apparatus 100, such as described in U.S. Pat. No. 10,028,759, and in U.S. Pat. No. 9,463,035, the entireties of which are incorporated by reference. The apparatus includes an inversion support catheter 107 and a flexible tube 103 that extends over the outer surface of the inversion catheter 107. The flexible tube 103 may be referred to as a tractor tube (or flexible tractor tube) and may be attached at one end region to a puller 101, which may be a pull wire or a pull tube (e.g., catheter), e.g., at the distal end region of the puller 101. In some examples the flexible tube 103 may be attached proximal to the distal end of the puller 101 (e.g. between 1 mm (0.04 inches) and 50 mm (1.97 inches) from the distal end, between 1 mm and 40 mm (1.57 inches), between 1 mm and 30 mm (1.18 inches), greater than 5 mm (0.20 inches), greater than 10 mm (0.39 inches), greater than 20 mm (0.79 inches), greater than 30 mm, etc. from the distal end of the puller). Pulling the puller 101 proximally inverts the flexible tube 103 over the distal end opening 111 of the inversion support catheter 107 to capture and remove a material (such as a clot) in the vessel lumen, as shown in FIGS. 1B and 1C. In operation, the amount of the material that may be captured corresponds to the length of the flexible tube 103.

In FIG. 1B, the inverting thrombectomy apparatus 100 is shown deployed near a clot 109. In the deployed configuration the puller 101 (shown here as a puller micro catheter, alternatively the puller 101 may be a wire) is held within an elongate inversion support catheter 107 so that the flexible tractor tube 103 extends from the end of the puller 101 and expands toward the inner radius of the elongate inversion support catheter 107. At the distal end opening 111 (designated in FIG. 1A) of the elongate inversion support catheter 107 the tractor tube 103 inverts over itself and extends proximally in an inverted configuration over the distal end of the elongate inversion support catheter 107. As shown in FIG. 1C, by pulling the puller 101 proximally, the tractor tube 103 inverts over the distal end opening 111 of the elongate inversion support catheter 107, inverting in a direction indicated by arrows 113 and 113′, drawing the adjacent clot 109 into the elongate inversion support catheter 107, as shown.

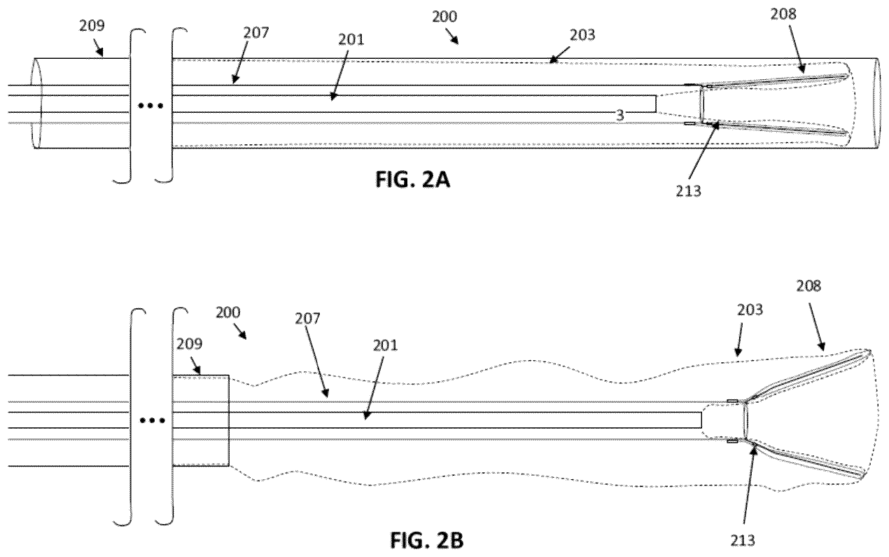

In FIG. 1A, the elongate inversion support catheter 107 is an elongate tube having a constant inner diameter, from proximal end to distal end. In some examples, the distal end of the inversion support catheter 107 may be funnel-shaped (or configured to expand into a funnel shape, see, e.g. FIGS. 2A-2B). In FIGS. 1A-1C, the inversion support catheter 107 is shown positioned between the tractor tube (e.g., flexible tube 103) and the puller 101 so that the flexible tube 103 can be pulled proximally by pulling on the puller 101 and rolling the flexible tube 103 into the elongate inversion support catheter 107 so that the flexible tube 103 inverts. The portion of the flexible tube 103 that is inverted over the distal end of the elongate inversion support catheter 107 has an outer diameter that is greater than the outer diameter of the elongate inversion support catheter 107.

The flexible tube 103 may be biased so that it has a relaxed expanded configuration with a diameter that is greater than the outer diameter (OD) of the elongate inversion support catheter 107. In addition, the flexible tube 103 may also be configured (e.g., by heat setting, etc.) so that when the flexible tube 103 is inverted and rolled over the distal end opening into the elongate inversion support catheter 107, the outer diameter of the flexible tube 103 within the elongate inversion support catheter 107 has an outer diameter that is about y times (y fold) the inner diameter of the elongate inversion support catheter 107 (e.g., where y is greater than 0.1×, 0.5×, 0.6×, 0.7×, 0.75×, 0.8×, 0.9×, 1×, etc. the inner diameter, ID, of the elongate inversion support catheter 107. This combination of an un-inverted diameter of the flexible tube 103 of greater than the diameter of the OD of the elongate inversion support catheter 107 and an inverted diameter of the flexible tube of greater than, e.g., 0.7× the ID of the elongate inversion support catheter 107 is surprisingly helpful for preventing jamming of the apparatus, both when deploying the apparatus and when rolling the flexible tube 103 over the distal end opening 111 of the elongate inversion support catheter 107 to draw in a clot. The flexible tube 103 may be expandable and may be coupled to the puller 101 as shown. In some examples the flexible tube 103 and the puller 101 may be made of the same material, but the flexible tube may be more flexible and/or expandable, or may be connected to elongate puller 101 (e.g., a push/pull wire or catheter). In some instances, the flexible tube 103 may be more flexible because the flexible tube 103 has a thinner material thickness relative to the elongate puller 101. In some instances, the flexible tube 103 may be more flexible than the elongate puller 101 because the flexible tube 103 may be made of a lower durometer version of a material while the elongate puller 101 is made of a higher durometer version of the same material. This is just an example. As mentioned above, the elongate puller 101 may be optional (e.g., the flexible tube 103 may itself be pulled proximally into the inversion support catheter 107).

As seen in FIG. 1C, the clot may be drawn into the elongate inversion support catheter 107 by pulling the flexible tube 103 proximally into the distal end of the elongate inversion support catheter 107, as indicated by the arrows 113, 113′ showing pulling of the inner portion of the flexible tube 103, resulting in rolling the flexible tube 103 over the distal end opening 111 of the catheter 107 and into the catheter distal end and inverting the expandable distal end region so that the flexible tube 103 is pulled into the inversion support catheter 107, shown by arrows 113 and 113′. The end of the flexible tube 103 outside of the inversion support catheter 107 may be loose relative to the outer wall of the inversion support catheter 107, meaning that there is sufficient clearance between the flexible tube 103 and the catheter 107 so that the flexible tube 103 easily moves relative to the inversion support catheter 107.

FIGS. 2A-2B illustrate an example of an inverting thrombectomy apparatus 200 that includes a funnel region at the distal end of an inversion support catheter 207. In this example, the inverting thrombectomy apparatus 200 includes an elongate, flexible inversion support catheter 207 that has an expandable funnel 208 at the distal end, shown in a collapsed configuration in FIG. 2A within an intermediate (e.g., delivery) catheter 209, and in an open configuration in FIG. 2B after being released from the intermediate catheter 209. The funnel 208 may be formed of a woven material and may be porous, particularly at a base region 213, where the funnel 208 extends from the inversion support catheter 207. A flexible tube 203 extends over the distal end (including the funnel 208) of the inversion support catheter 207 and inverts over the distal opening of the funnel 208. The flexible tube 203 may be, e.g., a knitted material, and may be biased to expand to an outer diameter (OD) that is larger than the OD of the funnel 208 in the open configuration. The flexible tube 203 is attached to a distal end region of a puller 201. The flexible tube (e.g., “tractor”) is attached to the distal end region of the puller 201. The funnel 208 may include two or more regions having different wall angles.

The apparatuses shown in FIGS. 1A-1C and 2A-2B may be modified or used with any of the methods and apparatuses described herein.

It can be appreciated that manipulating a thrombectomy system, such as the thrombectomy system 100 illustrated in FIGS. 1A-1B, it may be necessary for a clinician to grasp the inversion support catheter 107 and the puller 101 to pull the puller 101 proximally (relative to the inversion support catheter 107) to evert the flexible tube 103 over the distal end opening of the elongate inversion support catheter 107, thereby drawing a clot into the elongate inversion support catheter 107. Manipulating the thrombectomy system 200, as shown in FIGS. 2A-2B may require a clinician to grasp the inversion support catheter 207 and the puller 201 to pull the puller 201 proximally (relative to the inversion support catheter 207) to invert the flexible tube 203 over the distal end opening of the elongate inversion support catheter 207, thereby drawing a clot into the elongate inversion support catheter 207.

Further, in some instances, a clinician may inadvertently apply excess force to the inversion support catheter 107/207, the puller 101/201 or both the inversion support catheter 107/207 and the puller 101/201 when performing a thrombectomy procedure. It can be appreciated that applying an excessive amount of force (e.g., grasping the shafts of the inversion support catheter 107/207 and/or the puller 101/201 with excessive force) may result in the inadvertent kinking, bending, warping, etc. of the inversion support catheter 107/207 and/or the puller 101/201. Accordingly, it may be desirable for a clinician to utilize a separate assembly to apply a sufficient withdrawal force on the puller 101/201 when withdrawing the puller 101/201 proximally with respect to the inversion support catheter 107/207. Specifically, it may be desirable for a clinician to use an assembly to attach to the puller 101/201 such that the assembly engages an engagement member attached to the puller 101/201 to facilitate withdrawing the puller 101/201 proximally (with respect to the inversion support catheter 107/207) without damaging (e.g., kinking) either the puller 101/201 or the inversion support catheter 107/207. The following Figures provide a number of illustrative hand-held assemblies that may be used in pulling the puller 101/201 in order to move the flexible tube 103/203.

In some instances, an assembly that may be used for pulling the puller 101/201 may have an assembly body that is adapted to be held in a person's hand. In some instances, the assembly body may be ergonomic in design, meaning that the shape of the assembly body may be adapted to fit easily within a user's hand. In some instances, the assembly body may be adapted to provide one or more grasping surfaces that make it easier for the user to pull on the assembly body, thereby pulling on the puller 101/201. In some instances, the assembly may include features that make the assembly more intuitive to use, such as features that indicate which direction the user should pull the assembly body.

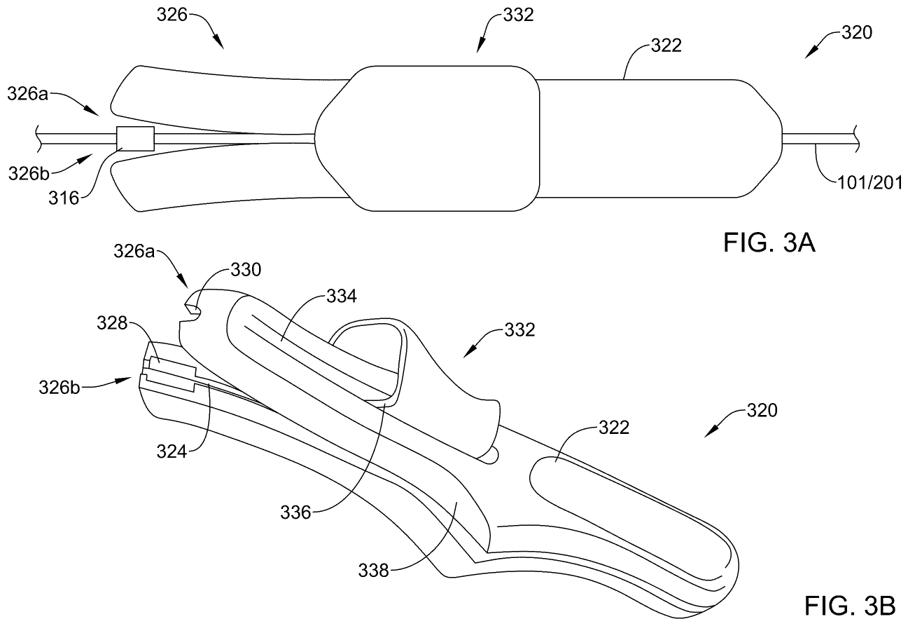

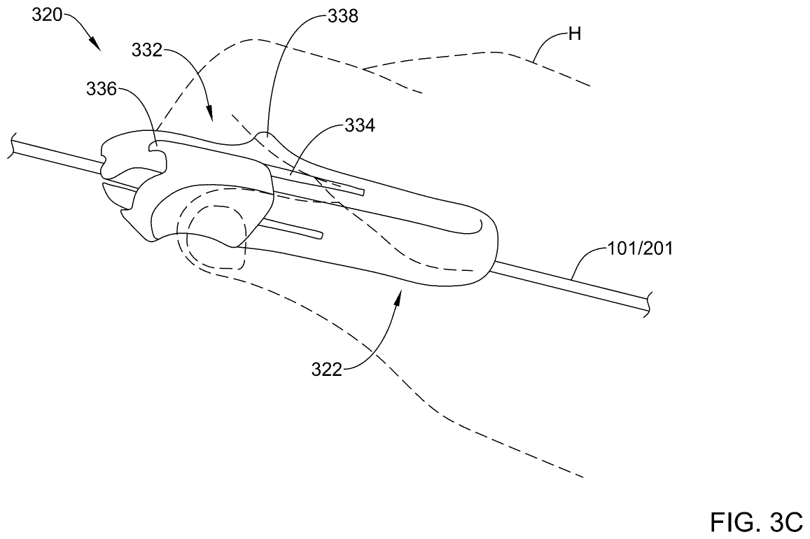

An assembly may include an engagement feature that is adapted to selectively engage an engagement member secured on a medical device shaft such as the puller 101/201. The engagement member may be a ring or other element that may be secured to the puller 101/201. As an example, an engagement member may be crimped onto the puller 101/201. As another example, an engagement member may be welded onto the puller 101/201. Soldering and adhesive are other possible attachment mechanisms that may be used. The engagement feature, which may include one element or multiple elements, may be movable between an open configuration in which the engagement feature does not engage the engagement member and a closed configuration in which the engagement feature engages the engagement member such that pulling on the assembly when the engagement feature is in the closed configuration results in a tensile force being applied to the puller 101/201. FIGS. 3 through 13B provide examples of illustrative hand-held assemblies having an engagement feature that is adapted to move between an open configuration and a closed configuration.

FIG. 3A is a top plan view of an illustrative assembly 320 shown in combination with the puller 101/201 extending through the assembly 320. FIG. 3B is a perspective view of the assembly 320 and FIG. 3C is a perspective view of the assembly 320 showing the assembly 320 within a user's hand and in combination with the puller 101/201. The assembly 320 may be considered as representing a slide embodiment. The assembly 320 includes an assembly body 322. A channel 324 extends through the assembly body 322 and allows the puller 101/201 to extend through the channel 324. As shown, the channel 324 extends through an interior of the assembly body 322. In some instances, at least part of the channel 324 may extend through an interior of the assembly body 322 and another part of the channel 324 may extend within an outer surface of the assembly body 322. As shown, an engagement member 316 is secured to the puller 101/201. In some instances, the engagement member 316 may be a metal collar or cylinder that is welded, soldered or adhesively secured to the puller 101/201. In some instances, the engagement member 316 may be a polymeric collar or cylinder that is adhesively secured to the puller 101/201.

The assembly 320 includes an engagement feature 326 that is adapted to selectively engage the engagement member 316. The engagement feature 326 may be movable between an open configuration in which the engagement feature 326 does not engage the engagement member 316 and a closed configuration in which the engagement feature 326 does engage the engagement member 316. The engagement feature 326 includes a first engagement feature element 326a that is molded into the assembly body 322 and a second engagement feature element 326b that is also molded into the assembly body 322. In some instances, the assembly body 322 may be formed of a resilient polymer that is easily deformable. As seen in FIG. 3A and 3B, the engagement feature 326 may be seen as being in an open configuration, with the first engagement feature element 326a and the second engagement feature element 326b being spaced apart. In some instances, the first engagement feature 326a and the second engagement feature 326b may be biased into this position.

As shown, the first engagement feature element 326a and the second engagement feature element 326b are symmetric to each other, and are each adapted to form an enlarged recess 328 that represents an enlargement of the channel 324 that is adapted to accommodate the engagement member 316 within the enlarged recess 328. Each of the first engagement feature 326a and the second engagement feature 326b also include a reduced diameter distal end 330 of the channel 324. Each enlarged recess 328 has a diameter that is greater than a diameter of the engagement member 316 and each reduced diameter distal end 330 has a diameter that is less than diameter of the engagement member 316. As a result, capturing the engagement member 316 within each enlarged recess 328 with the first engagement feature element 326a and the second engagement feature element 326b urged towards each other means that pulling on the assembly body 322 results in a force being applied to the puller 101/201.

The engagement feature 326 also includes a slide 332 that is adapted to be moved back and forth relative to the assembly body 322 in order to selectively allow the first engagement feature element 326a and the second engagement feature element 326b to remain apart, in the open configuration (as seen in FIGS. 3A and 3B), or to urge the first engagement feature element 326a and the second engagement feature element 326b towards each other (as seen in FIG. 3C). In some instances, the assembly body 322 may include tracks 334 (only one side is visible) that are formed within the assembly body 322 to accommodate corresponding sliders 336 that are formed as part of the slide 332. As can be seen for example in FIG. 3C, the assembly 320 is disposed within a user's hand H. In some instances, a person may use their thumb to push the slide 332 distally, thereby moving the first engagement feature element 326a and the second engagement feature element 326b towards each other, thereby moving the engagement feature 326 from the open configuration to the closed configuration. In some instances, assembly body 322 may include ergonomic features such as a finger grip 338 that allows the person to more easily grip the assembly body 322.

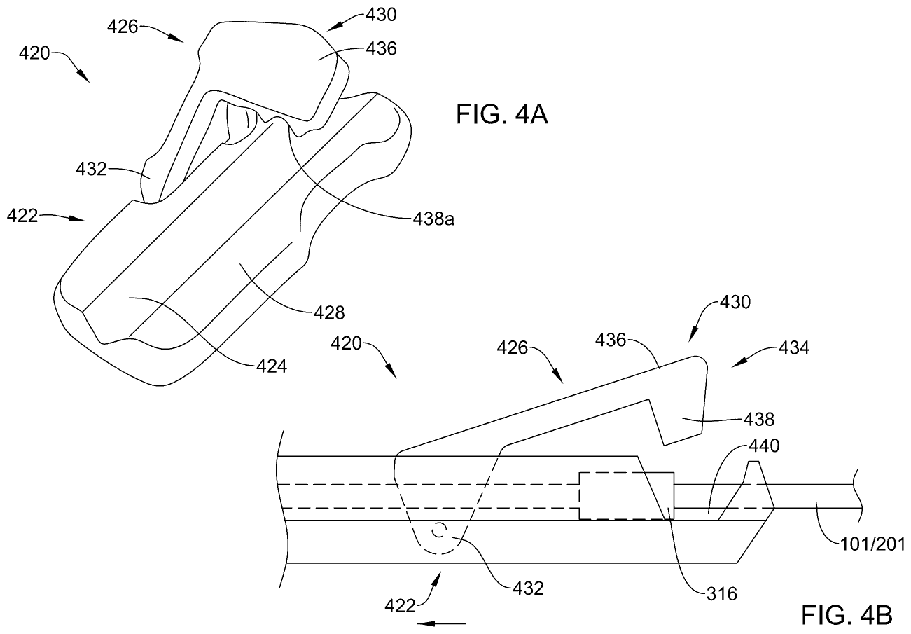

FIG. 4A is a perspective view of an illustrative assembly 420 that may be used in combination with the puller 101/201. FIG. 4B is a side view of the assembly 420. The assembly 420 includes an assembly body 422. The assembly 420 may be considered as representing a lever embodiment. A channel 424 extends along an outer surface 428 of the assembly body 422. While the channel 424 is shown as extending in its entirety within the outer surface 428 of the assembly body 422, in some instances, at least part of the channel 424 may extend through an interior of the assembly body 422. As shown in FIG. 4B, the engagement member 316 is secured to the puller 101/201.

The assembly 420 includes an engagement feature 426 that is adapted to selectively engage the engagement member 316. The engagement feature 426 may be movable between an open configuration in which the engagement feature 426 does not engage the engagement member 316 and a closed configuration in which the engagement feature 426 does engage the engagement member 316. As shown, the engagement feature 426 includes a lever 430 including a first end 432 that is pivotably attached to a side of the assembly body 422 and an opposing second end 434 that includes a thumb surface 436 and a tab 438 that extends away form the thumb surface 436 towards the channel 424. The tab 438 may include a recess 438a that is sized to allow the puller 101/201 to pass through but to not allow the engagement member 316 to pass through. In some instances, the engagement feature 426 also includes a recessed region 440 proximate the channel 424 that the tab 438 is able to fit into. While FIG. 4B also shows the engagement feature 426 in the open configuration, it can be seen that by pushing the lever 430 downward, with the tab 438 extending into the recessed region 440, and pulling the assembly 420 in a direction indicated by an arrow 442 will result in the tab 438 engaging the engagement member 316 and pulling the puller 101/201 in the direction indicated by the arrow 442.

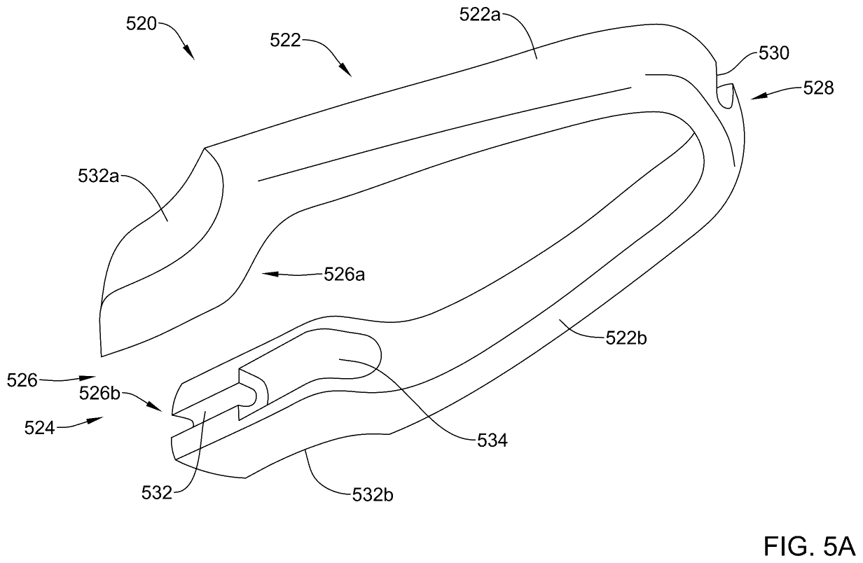

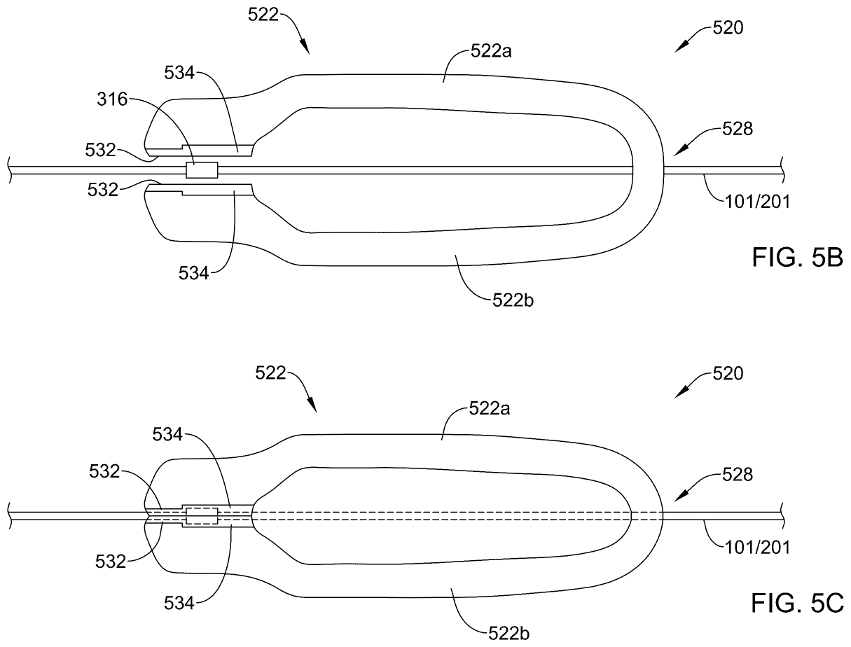

FIG. 5A is a perspective view and FIG. 5B is a side view of an illustrative assembly 520, shown in an open configuration. FIG. 5C is a side view of the illustrative assembly 520, shown in a closed configuration. The assembly 520 may be considered as representing a tweezer embodiment. The assembly 520 includes an assembly body 522. The assembly body 522 includes a first assembly body half 522a extending from an assembly body midpoint 528 and a second assembly body half 522b extending from the assembly body midpoint 528. In some instances, the assembly body midpoint 528 may include a recess 530 that is dimensioned to allow the puller 101/201 to pass through the recess 530. The first assembly body half 522a includes a first finger grip surface 532a and the second assembly body half 522b includes a second finger grip surface 532b. In some instances, the first finger grip surface 532a and the second finger grip surface 532b may double as pull locations, easily showing where a user should grasp the assembly 520.

The assembly 520 includes an engagement feature 526 that is adapted to selectively engage the engagement member 316. The engagement feature 526 may be movable between an open configuration in which the engagement feature 526 does not engage the engagement member 316 and a closed configuration in which the engagement feature 526 does engage the engagement member 316. The engagement feature 526 includes a first engagement feature element 526a that is molded into the first assembly body half 522a and a second engagement feature element 526b that is molded into the second assembly body half 522b. In some instances, the assembly body 522 may be formed of a resilient polymer that is easily deformable. In some instances, the assembly body 522 is biased into the open configuration shown for example in FIGS. 5A and 5B, but is easily squeezed into the closed configuration.

Each of the first engagement feature element 526a and the second engagement feature element 526b includes a first recess 532 that is dimensioned to accommodate the puller 101/201 but not accommodate the engagement member 316. Each of the first engagement feature element 526a and the second engagement feature element 526b also include a second recess 534 that has a larger diameter than the first recess 532 such that the second recess 534 is able to accommodate the engagement member 316. In some instances, the first recess 532 and the second recess 534, particularly when the engagement feature 526 is in the closed configuration, as shown in FIG. 5C, may be considered as being axially aligned with the recess 530 formed within the assembly body midpoint 528. Each of the first recesses 532 and the second recesses 534 together may be considered as forming a channel 524. In some instances, the channel 524 may be considered as also extending through the recess 530 disposed within the assembly body midpoint 528.

In some instances, the first engagement feature element 526a and the second engagement feature element 526b may be considered as being biased into an open configuration in which the first engagement feature element 526a and the second engagement feature element 526b, and of course the first assembly body half 522a and the second assembly body half 522b are spaced apart from each other. To use, the user may align the puller 101/201 with the recess 530 disposed within the assembly body midpoint 528. Subsequently grasping and squeezing the first assembly body half 522a and the second assembly body half 522b together causes the first engagement feature element 526a and the second engagement feature element 526b together into the closed configuration. The user can then pull on the assembly 520 to exert a force on the puller 101/201.

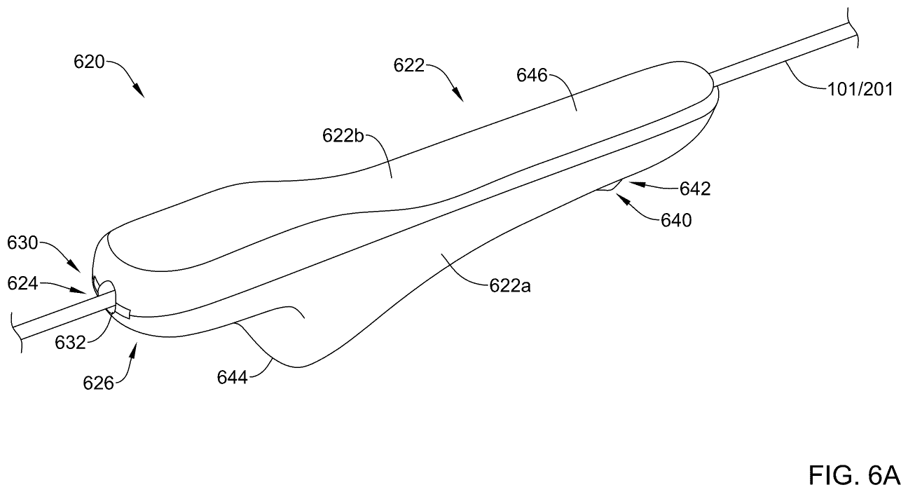

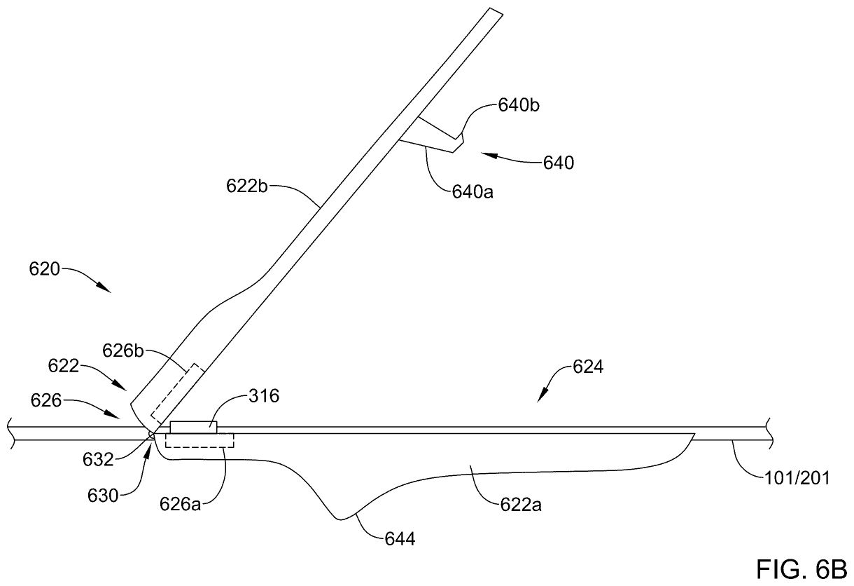

FIG. 6A is a perspective view of an illustrative assembly 620 in combination with the puller 101/201, shown in a closed configuration. FIG. 6B is a side view of the illustrative assembly 620, shown in an open configuration. The assembly 620 may be considered as representing a fold and pull embodiment. The assembly 620 includes an assembly body 622. A channel 624 extends through the assembly body 622 and allows the puller 101/201 to extend through the channel 624. The assembly body 622 includes a main body portion 622a and a lid portion 622b that is hingedly coupled to the main body portion 622a via a hinge 630. In some instances, the hinge 630 is a living hinge, meaning that the hinge 630 is formed of the same material as the main body portion 622a and the lid portion 622b, and the material is sufficiently malleable to allow the lid portion 622b to repeatedly pivot relative to the main body portion 622a.

The assembly 620 includes an engagement feature 626 that is adapted to selectively engage the engagement member 316. The engagement feature 626 may be movable between an open configuration in which the engagement feature 626 does not engage the engagement member 316 and a closed configuration in which the engagement feature 626 does engage the engagement member 316. The engagement feature 626 includes a first engagement feature element 626a that is molded into the main body portion 622a and a second engagement feature element 626b that is molded into the lid portion 622b. In some instances, the first engagement feature element 626a and the second engagement feature element 626b each include recesses formed within the channel 624 that have a diameter greater than a diameter of the engagement member 316. The channel 624 is dimensioned to accommodate the puller 101/201 extending through the channel 624. In some instances, the channel 624 has a distal end 632 dimensioned to accommodate the puller 101/201 but not the engagement member 316. With the puller 101/201 disposed within the channel 624, and the engagement member 316 disposed within the engagement feature 626, a user can pull the assembly 620 in a left to right direction (in the illustrated orientation) in order to exert a pulling force on the puller 101/201.

In some instances, the assembly 620 includes a release mechanism 640 extending down from the lid portion 622b. The release mechanism 640 includes a member 640a having a hooked end 640b. When the assembly 620 is in the closed configuration as shown in FIG. 6A, the release mechanism 640 engages a release aperture 642 in order to secure the lid portion 622b in position relative to the main body portion 622a. Because the release mechanism 640 extends out the bottom of the main body portion 622a, a user is able to push on the release mechanism 640 to release the release mechanism 640 from the release aperture 642, and thus release the lid portion 622b from the main body portion 622a to allow reloading of the puller 101/201 within the assembly 620.

In some instances, the assembly body 622 may include one or more features that render the assembly body 622 more ergonomic. As an example, the assembly body 622 may include a trigger region 644 that allows the user to hook a finger around the trigger region 644 to more easily exert a pulling force on the assembly body 622, and hence more easily exert a pulling force on the puller 101/201. In some instances, an upper surface 646 of the lid portion 622b may also be shaped to more naturally accommodate the user's hand as the user wraps their hand around the assembly body 622. In some instances, the first engagement feature element 626a and the second engagement feature element 626b are each disposed proximate the hinge 630. In some instances, the hinge 630 may not be a living hinge.

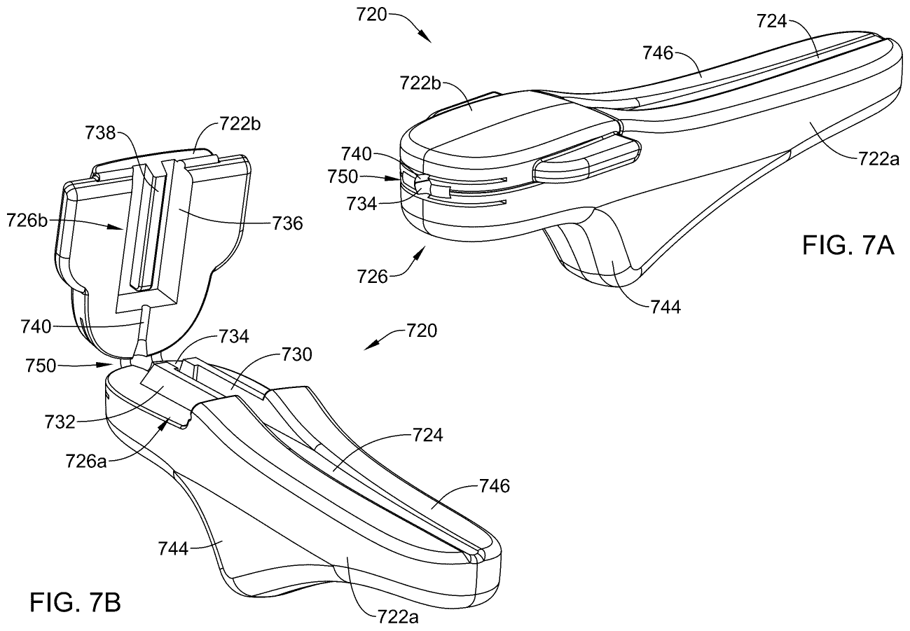

FIG. 7A is a perspective view of an illustrative assembly 720, shown in a closed configuration. FIG. 7B is a side view of the illustrative assembly 720, shown in an open configuration. The assembly 720 may be considered as representing another fold and pull embodiment. The assembly 720 includes an assembly body 722. A channel 724 extends through the assembly body 722 and allows the puller 101/201 to extend through the assembly 720. The assembly body 722 includes a main body portion 722a and a lid portion 722b that is hingedly coupled to the main body portion 722a via a hinge 750. In some instances, the hinge 750 is a living hinge, meaning that the hinge 750 is formed of the same material as the main body portion 722a and the lid portion 722b, and the material is sufficiently malleable to allow the lid portion 722b to repeatedly pivot relative to the main body portion 722a. In some instances, the hinge 750 may not be a living hinge.

The assembly 720 includes an engagement feature 726 that is adapted to selectively engage the engagement member 316. The engagement feature 726 may be movable between an open configuration in which the engagement feature 726 does not engage the engagement member 316 and a closed configuration in which the engagement feature 726 does engage the engagement member 316. The engagement feature 726 includes a first engagement feature element 726a that is molded into the main body portion 722a and a second engagement feature element 726b that is molded into the lid portion 722b. In some instances, the hinge 750 may be disposed adjacent the engagement feature 726.

In some instances, the first engagement feature element 726a includes a recess 730 that is axially aligned with the channel 724. The recess 730 is defined in part by walls 732 and terminates at a reduced diameter exit 734 at a distal end of the recess 730. The second engagement feature element 726b includes a recess 736 that is adapted to accommodate the walls 732 defining the recess 730. The second engagement feature element 726b also includes a projection 738 that extends within the recess 736 and is adapted to hold the engagement member 316 down within the recess 730 when the puller 101/201 is disposed within the channel 724. The second engagement feature element 726b also includes a reduced diameter channel 740 that is axially aligned with the projection 738 and the recess 730 in order to accommodate the puller 101/201 distal of the engagement feature 316. With the puller 101/201 disposed within the channel 724, and the engagement member 316 disposed within the engagement feature 726, a user can pull the assembly 720 in a right to left direction (in the illustrated orientation) in order to exert a pulling force on the puller 101/201.

In some instances, the assembly body 722 may include one or more features that render the assembly body 722 more ergonomic. As an example, the assembly body 722 may include a trigger region 744 that allows the user to hook a finger around the trigger region 744 to more easily exert a pulling force on the assembly body 722, and hence more easily exert a pulling force on the puller 101/201. In some instances, an upper surface 746 of the body portion 722a may also be shaped to more naturally accommodate the user's hand as the user wraps their hand around the assembly body 722.

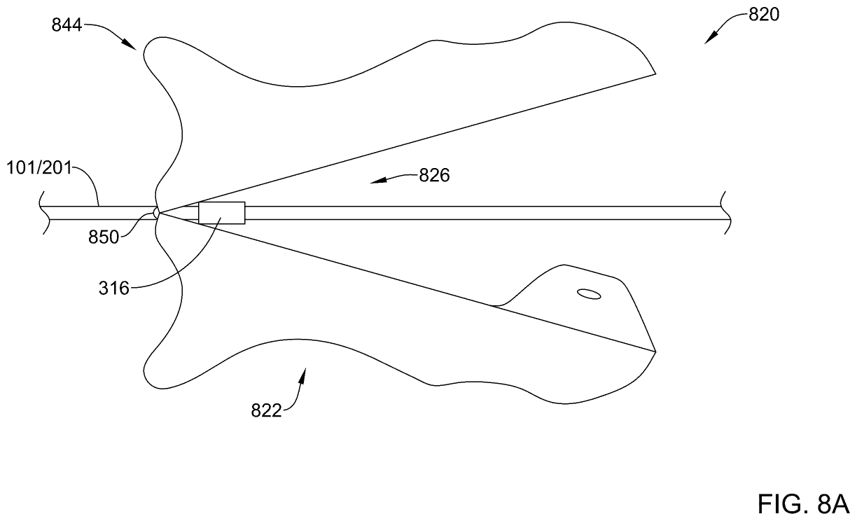

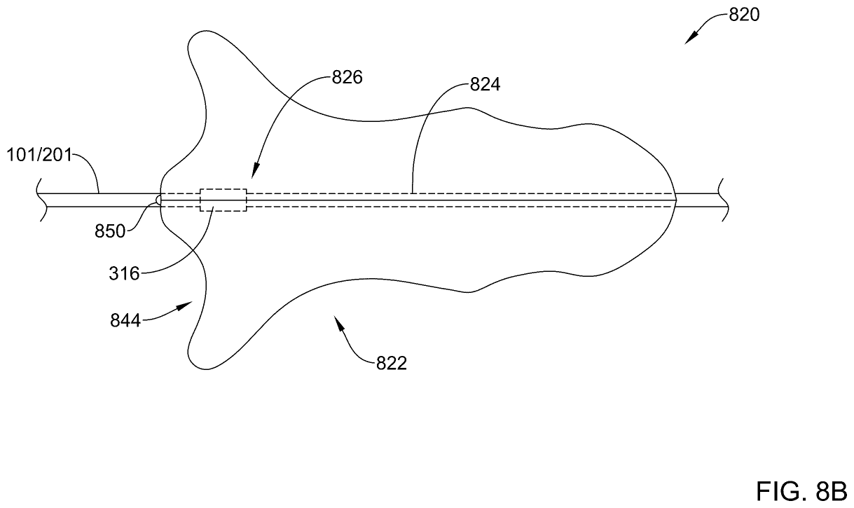

FIG. 8A is a side view of an illustrative assembly 820, shown in combination with the puller 101/201 and in an open configuration. FIG. 8B is a side view of the assembly 820, shown in combination with the puller 801/802 and in a closed configuration. The assembly 820 may be considered as representing another fold and pull embodiment. The assembly 820 includes an assembly body 822. A channel 824 extends through the assembly body 822 and allows the puller 101/201 to extend through the assembly 820. The assembly body 822 includes a first body portion 822a and a second body portion 822b that is hingedly coupled to the first body portion 822a via a hinge 850. In some instances, the hinge 850 is a living hinge, meaning that the hinge 850 is formed of the same material as the first body portion 822a and the second body portion 822b, and the material is sufficiently malleable to allow the second body portion 822b to repeatedly pivot relative to the first body portion 822a. In some instances, the hinge 850 may not be a living hinge.

The assembly 820 includes an engagement feature 826 that is adapted to selectively engage the engagement member 316. The engagement feature 826 may be movable between an open configuration in which the engagement feature 826 does not engage the engagement member 316 and a closed configuration in which the engagement feature 826 does engage the engagement member 316. The engagement feature 826 includes a first engagement feature element 826a that is molded into the first body portion 822a and a second engagement feature element 826b that is molded into the second portion 822b. In some instances, the hinge 850 may be disposed adjacent the engagement feature 826.

In some instances, the first engagement feature element 826a includes a first recess that is aligned with the channel 824 and a second recess that is aligned with the channel 824. The first recess may be dimensioned to accommodate the engagement member 316 and the second recess may have a smaller diameter that accommodates the puller 101/201 to pass through, but does not allow the engagement member 316 to pass through. In some instances, the second engagement feature element 826b includes a first recess that is aligned with the channel 824 and a second recess that is aligned with the channel 824. The first recess may be dimensioned to accommodate the engagement member 316 and the second recess may have a smaller diameter that accommodates the puller 101/201 to pass through, but does not allow the engagement member 316 to pass through. With the puller 101/201 disposed within the channel 824, and the engagement member 316 disposed within the engagement feature 826, a user can pull the assembly 820 in a left to right direction (in the illustrated orientation) in order to exert a pulling force on the puller 101/201.

In some instances, the assembly body 822 may include one or more features that render the assembly body 822 more ergonomic. As an example, the assembly body 822 may include a gripping region 844 that allows the user to hook several fingers around the gripping region 844 to more easily exert a pulling force on the assembly body 822, and hence more easily exert a pulling force on the puller 101/201. In some instances, the assembly body 822 may also be shaped to more naturally accommodate the user's hand as the user wraps their hand around the assembly body 822.

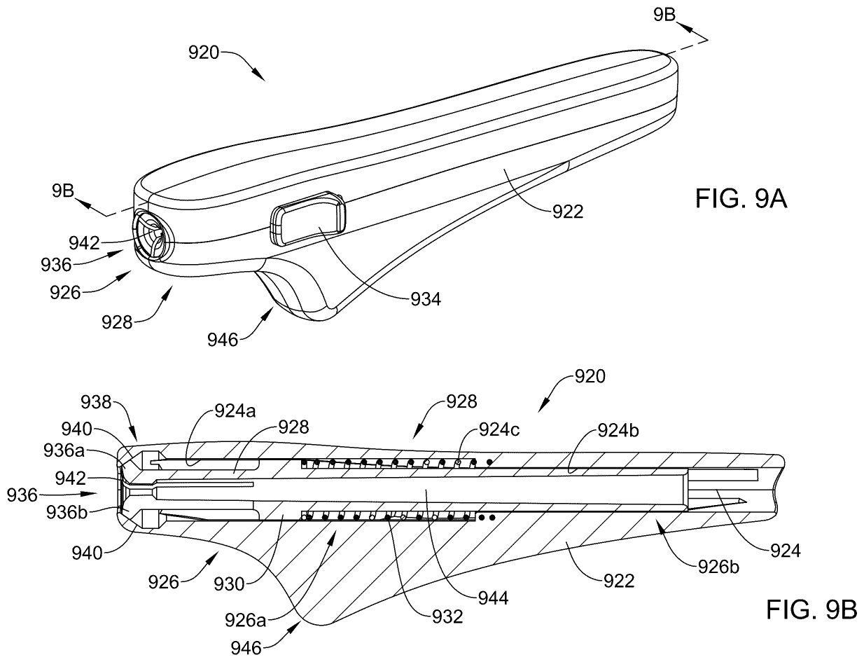

FIG. 9A is a perspective view of an illustrative assembly 920 shown in a closed configuration and FIG. 9B is a cross-sectional view taken along the line 9B-9B in FIG. 9A. The assembly 920 may be considered as being an auto-lock embodiment. The assembly 920 includes an assembly body 922 that defines a channel 924 extending through the assembly body 922. The channel 924 allows the puller 101/201 to pass through the assembly 920. The channel 924 also accommodates an engagement feature 926 that fits within the channel 924. In some instances, the channel 924 includes a distal section 924a and a proximal section 924b. The distal section 924a may have a larger diameter than the proximal section 924b, and may be adapted to accommodate a larger diameter portion of an engagement feature 926 while the proximal section 924b may be adapted to accommodate a smaller diameter portion of the engagement feature 926.

The engagement feature 926 includes a distal segment 926a that fits into the distal section 924a of the channel 924 and a proximal segment 926b that fits into the proximal section 924b of the channel 924. The engagement feature 926 may be movable between an open configuration in which the engagement member 316 is free to move relative to the engagement feature 926 and a closed configuration in which the engagement member 316 is captured by the engagement feature 926.

The engagement feature 926 includes a collet 928. The collet 928 includes an enlarged diameter region 930 that is dimensioned to fit within the distal section 924a of the channel 924. A spring 932 extends between the enlarged diameter region 930 and a proximal end 924c of the distal section 924a of the channel 924. In some instances, the engagement feature 926 may include sliders 934 (one on each side of the assembly body 922) that are operably coupled to the enlarged diameter region 930 such that a user can manually compress the spring 932 by moving the sliders 934 in a proximal direction, thereby moving the collet 928 proximally within the channel 924. The spring 932 biases the collet 928 into the closed configuration, in which the engagement member 316 is trapped within the collet 928. Moving the collet 928 proximally within the channel 924 moves the collet 928 into the open configuration.

In some instances, the sliders 934 may be shaped to more easily enable a user to push the sliders 934 with their thumb, for example. In some instances, the sliders 934 may have a chevron design, or perhaps an arrow, molded into the sliders 934 to provide an indication of which direction the user should move the sliders 934. In some instances, the assembly body 922 may include markings that provide an indication of which direction the user should move the sliders 934. In some instances, the assembly body 922 may include an elongate slot under each of the sliders 934 to provide an indication of directionality. These are just examples.

A distal end of the collet 928 may be seen as having an enlarged diameter, and to be axially divided into several segments 936. The distal end may be divided into two, three, four or more collet segments 936, although two segments 936a and 936b are easily visible in FIG. 9B. The distal section 924a of the channel 924 includes an enlarged diameter region 938 defined by ramp surfaces 940. The collet segments 936 define a passage 942 that is dimensioned to accommodate the puller 101/201 but not the engagement member 316. As the collet 928 is moved proximally, either by manually engaging the sliders 934 or by pulling an engagement member 316 proximally into the assembly 920, the collet segments 936 are able to move radially outwardly within the enlarged diameter region 938, thereby allowing the engagement member 316 to pass through the temporarily expanded passage 942 and reach an internal passage 944 within the collet 928. Once the engagement member 316 is disposed within the internal passage 944 within the collet 928, a user is able to exert a force on the puller 101/201 by pulling the assembly 920 in a proximal direction.

In some instances, the assembly 920 may be used in procedures intended for removing a thrombus or clot (such as the clot 109) from either an arterial location or a venous location. In some instances, the puller 101/201 may have a single engagement member 316 secured to the puller 101/201, particularly for arterial applications. In some instances, the puller 101/201 may include two or more engagement members 316 spaced apart from each other and each secured to the puller 101/201, particularly for venous applications. As shown in FIG. 9B, the internal passage 944 is adapted for venous applications, as the internal passage 944 is adapted to accommodate more than one engagement member 316 within the internal passage 944. In some instances, particularly for arterial applications in which the puller 101/201 only has a single engagement member 316, the internal passage 944 may include a stop that allows the engagement member 316 to enter the internal passage 944 but not proceed down the internal passage 944. In some instances, this may provide a more positive engagement with the engagement member 316, which can reduce or even eliminate the possibility of the engagement member 316 moving within the internal passage 944 if the assembly 920 is set down during a procedure.

In some instances, the assembly body 922 may include one or more features that render the assembly body 922 more ergonomic. As an example, the assembly body 922 may include a trigger region 946 that allows a user to hook a finger around the trigger region 946, and hence more easily exert a pulling force on the puller 101/201. In some instances, the assembly body 922 may also be shaped to more naturally accommodate the user's hand as the user wraps their hand around the assembly body 922.

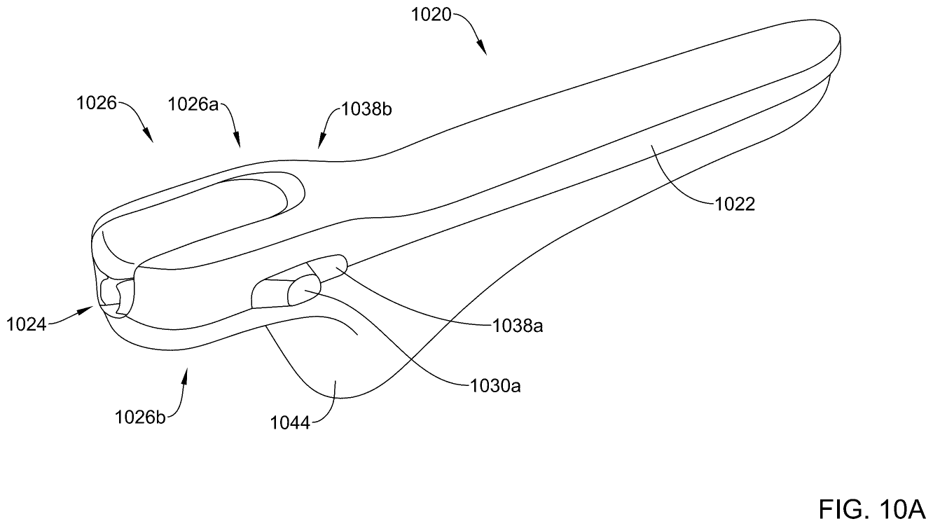

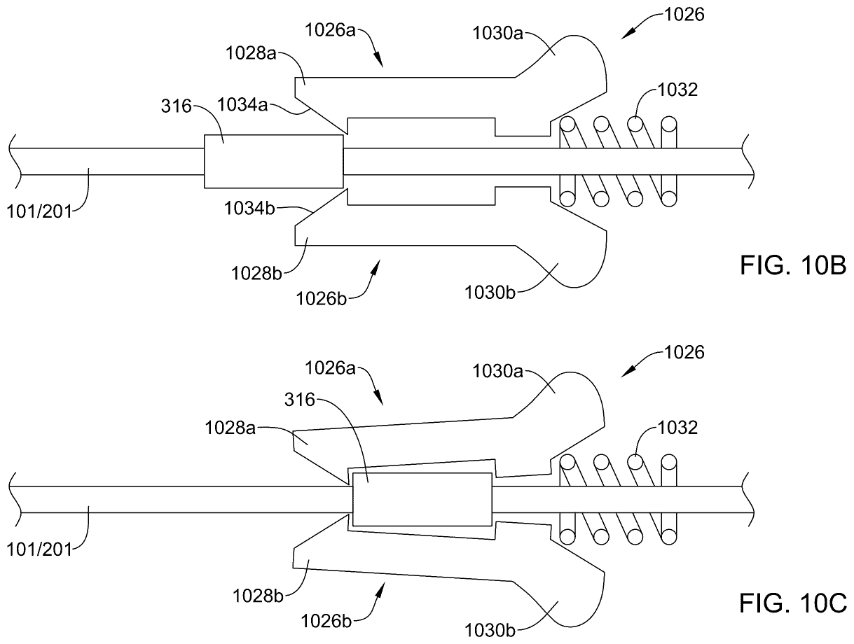

FIG. 10A is a perspective view of an illustrative assembly 1020, shown in a closed configuration. The assembly 1020 may be considered as being another auto-lock embodiment. In some instances, the assembly 1020 may be used in procedures intended for removing a thrombus or clot (such as the clot 109) from either an arterial location or a venous location. In some instances, the puller 101/201 may have a single engagement member 316 secured to the puller 101/201, particularly for arterial applications. In some instances, the puller 101/201 may include two or more engagement members 316 spaced apart from each other and each secured to the puller 101/201, particularly for venous applications.

The assembly 1020 includes an assembly body 1022 that defines a channel 1024 extending through the assembly body 1022. The channel 1024 allows the puller 101/201 to pass through the assembly 1020. The channel 1024 also accommodates an engagement feature 1026 that fits within the channel 1024. The engagement feature 1026 may be movable between an open configuration in which the engagement member 316 is free to move relative to the engagement feature 1026 and a closed configuration in which the engagement member 316 is captured by the engagement feature 1026. FIG. 10B is a side view of the engagement feature 1026 with the engagement member 316 exterior to the engagement feature 1026 and FIG. 10C is a side view of the engagement feature 1026 with the engagement member 316 captured within the engagement feature 1026.

The engagement feature 1026 includes a first engagement feature element 1026a and a second engagement feature element 1026b. The first engagement feature element 1026a may be considered as being disposed above (in the illustrated orientation shown in FIG. 10A) the second engagement feature element 1026a. The first engagement feature element 1026a includes a first jaw portion 1028a and a first handle portion 1030a. The second engagement feature 1026b includes a second jaw portion 1028b and a second handle portion 1030b. In some instances, the first jaw portion 1028a and the second jaw portion 1028b are biased into contact with each other. As shown, the engagement feature 1026 may include a biasing member such as a spring 1032. The spring 1032 is adapted to bias the first engagement feature element 1026a and the second engagement feature element 1026b into the closed configuration in which the first jaw portion 1028a and the second jaw portion 1028b close around the engagement member 316.

The first jaw portion 1028a includes a first ramp portion 1034a and the second jaw portion 1028b includes a second ramp portion 1034b. Together, the first ramp portion 1034a and the second ramp portion 1034b allow the engagement member 316 to urge the first jaw portion 1028a and the second jaw portion 1028b away from their biased position such that the engagement member 316 can be pushed into engagement with the engagement feature 1026. With respect to FIG. 10B, as the engagement member 316 is urged into contact with the engagement feature 1026, the engagement member 316 exerts a force on the first ramp portion 1034a and the second ramp portion 1036b. Because of the angled surfaces provided by the first ramp portion 1034a and the second ramp portion 1036b, a portion of the exerted force pushes in an outward direction on the first ramp portion 1034a and the second ramp portion 1034b. This causes the first jaw portion 1028a and the second jaw portion 1028b to move away from each other. Continuing to exert a force on the engagement member 316 may result in the engagement member 316 moving to a position in which the spring 1032 urges the first engagement feature element 1026a and the second engagement feature element 1026b into contact with each other, thereby capturing the engagement member 316 between the first engagement feature element 1026a and the second engagement feature element 1026b, as shown in FIG. 10C.

In some instances, the assembly body 1022 may include a first side aperture 1038a and a second side aperture 1038b, although only the first side aperture 1038 is directly visible in the orientation shown in FIG. 10A. In some instances, the first handle portion 1030a of the first engagement member feature 1026a may extend out the first side aperture 1038a and the second handle portion 1030b of the second engagement member feature 1026b may extend out the second side aperture 1038b. It will be appreciated that this allows a user to manually move the engagement feature 1026 from the closed configuration to the open configuration. In some instances, the engagement feature 1026 may be well-suited for releasing a first engagement member 316 and engaging a second or subsequent engagement member 316, particularly in a venous application.

In some instances, the assembly body 1022 may include one or more features that render the assembly body 1022 more ergonomic. As an example, the assembly body 1022 may include a trigger region 844 that allows the user to hook a finger around the trigger region 1044 to more easily exert a pulling force on the assembly body 1022, and hence more easily exert a pulling force on the puller 101/201. In some instances, the assembly body 1022 may also be shaped to more naturally accommodate the user's hand as the user wraps their hand around the assembly body 1022.

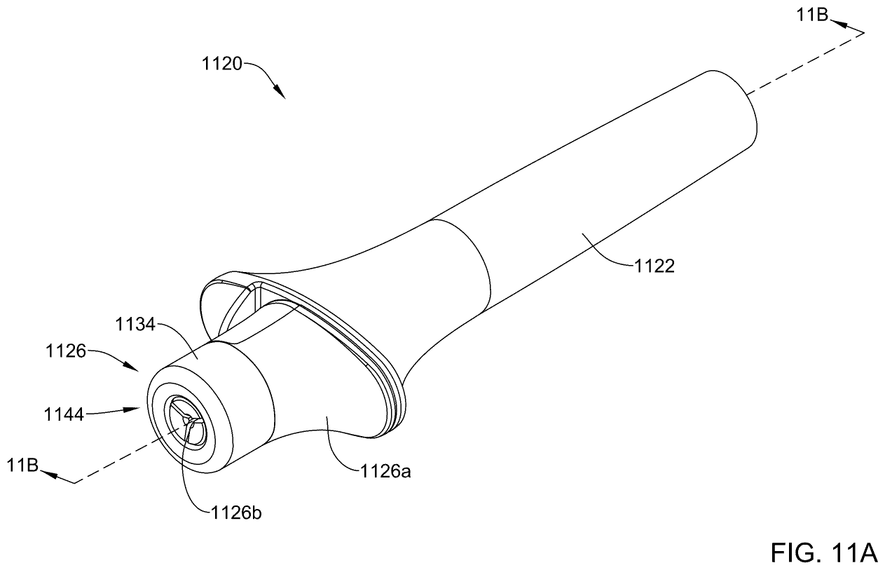

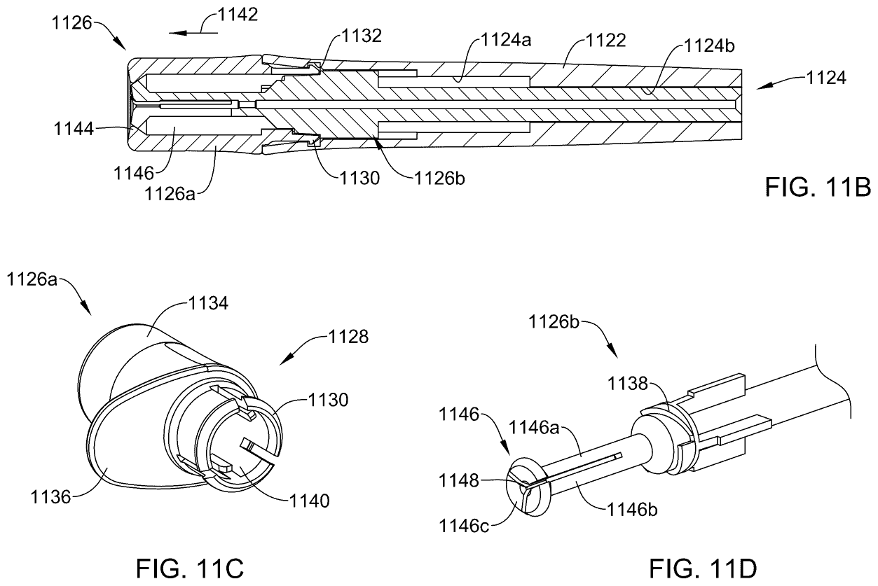

FIG. 11A is a perspective view of an illustrative assembly 1120, shown in a closed configuration, and FIG. 11B is a cross-sectional view taken along the line 11B-11B in FIG. 11A. The illustrative assembly 1120 may be considered as being a twist embodiment. The assembly 1120 includes an assembly body 1122 that defines a channel 1124 extending through the assembly body 1122. The channel 1124 allows the puller 101/201 to pass through the assembly 1120. The channel 1124 also accommodates an engagement feature 1126 that is partially disposed distal of the assembly body 1122 and is partially disposed within the channel 1124. In some instances, the channel 1124 includes a distal section 1124a and a proximal section 1124b. The distal section 1124a may have a larger diameter than the proximal section 1124b, and may be adapted to accommodate a larger diameter portion of an engagement feature 1126 while the proximal section 1124b may be adapted to accommodate a smaller diameter portion of the engagement feature 1126.

The engagement feature 1126 may be movable between an open configuration in which the engagement member 316 is free to move relative to the engagement feature 1126 and a closed configuration in which the engagement member 316 is captured by the engagement feature 1126. The engagement feature 1126 includes a first engagement feature element 1126a and a second engagement feature element 1126b. FIG. 11C provides a perspective view of the first engagement feature element 1126a and FIG. 11D provides a perspective view of the second engagement feature element 1126b. The first engagement feature element 1126a is disposed adjacent to the assembly body 1122. The second engagement feature element 1126b is partially disposed within the distal section 1124a of the channel 1124, and extends both distally out of the assembly body 1122, into an interior of the first engagement feature element 1126a, and proximally into the proximal section 1124b of the channel 1124.

The first engagement feature element 1126a includes an annular extension 1128 that extends proximally from the first engagement feature element 1126a and into the distal section 1124a of the channel 1124. The annular extension 1128 includes an annular ring 1130 that is adapted to engage a corresponding annular recess 1132 formed within the assembly body 1122 proximate the channel 1124. The annular ring 1130 may form a snap-fit with the annular recess 1132 in order to secure the first engagement feature element 1126a relative to the assembly body 1122 while allowing the first engagement feature element 1126a to rotate relative to the assembly body 1122. In some instances, this prevents the assembly 1120 from falling apart during use. The first engagement feature element 1126a includes a housing 1134 and a widened portion 1136. The widened portion 1136 allows a user to manually rotate the first engagement feature element 1126a relative to the assembly body 1122 in order to move the engagement feature 1126 from the closed configuration to the open configuration.

The second engagement feature element 1126b includes a threaded surface 1138 that threadedly engages a corresponding threaded surface 1140 disposed within the annular extension 1128 of the first engagement feature element 1126a. As a result, rotating the first engagement feature element 1126a relative to the second engagement feature element 1126b (and hence relative to the assembly body 1122) causes the first engagement feature element 1126a to translate relative to the second engagement feature element 1126b in a direction indicated by an arrow 1142 as shown in FIG. 11B. The first engagement feature element 1126a includes a distal opening 1144. As seen in FIG. 11B, the housing 1134 defines an interior volume 1146 that has a diameter that increases proximally from a minimum diameter at the distal opening 1144. With the first engagement feature element 1126a displaced distally, the distal end of the second engagement feature element 1126b is free to expand, thereby allowing the engagement member 316 to pass through into a larger diameter recess within the second engagement feature element 1126b.

The second engagement feature element 1126b includes a distal end 1146 having an enlarged diameter, and is axially divided into several segments 1146a, 1146b and 1146c. It will be appreciated that in the closed configuration (as shown in FIG. 11B), the distal opening 1144 of the first engagement feature element 1126a will hold the segments 1146a, 1146b and 1146c together as shown, defining a small aperture 1148 therebetween that is dimensioned to allow the puller 101/201 to pass through, but will not allow the engagement member 316 to pass through. By moving the second engagement feature element 1126b proximally relative to the first engagement member element 1126a, the segments 1146a, 1146b and 1146c are free to move radially outwardly, temporarily increasing the diameter of the aperture 1148 to a point that the engagement member 316 is able to pass through the aperture 1148 and reach a larger diameter recess within the second engagement feature element 1126b that is dimensioned to accommodate the engagement member 316. Once the engagement member 316 is disposed within the recess within the second engagement feature element 1126b, a user is able to exert a force on the puller 101/201 by pulling the assembly 1120 in a proximal direction.

In some instances, the assembly body 1122 may include one or more features that render the assembly body 1122 more ergonomic. As an example, the assembly body 1122 and the housing 1134 may together provide a surface that allows a user to pull proximally on the assembly body 1122. In some instances, the more proximal portions of the assembly body 1122 may also be shaped to more naturally accommodate the user's hand as the user wraps their hand around the assembly body 1122.

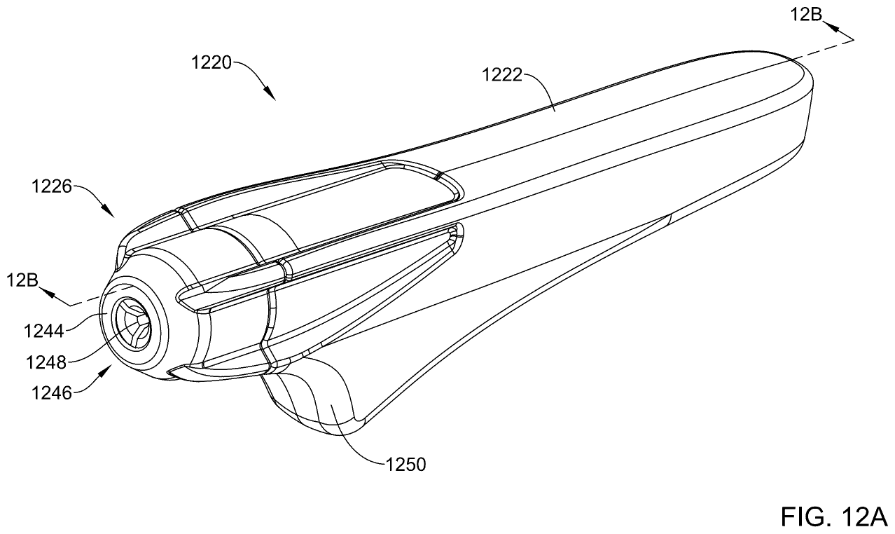

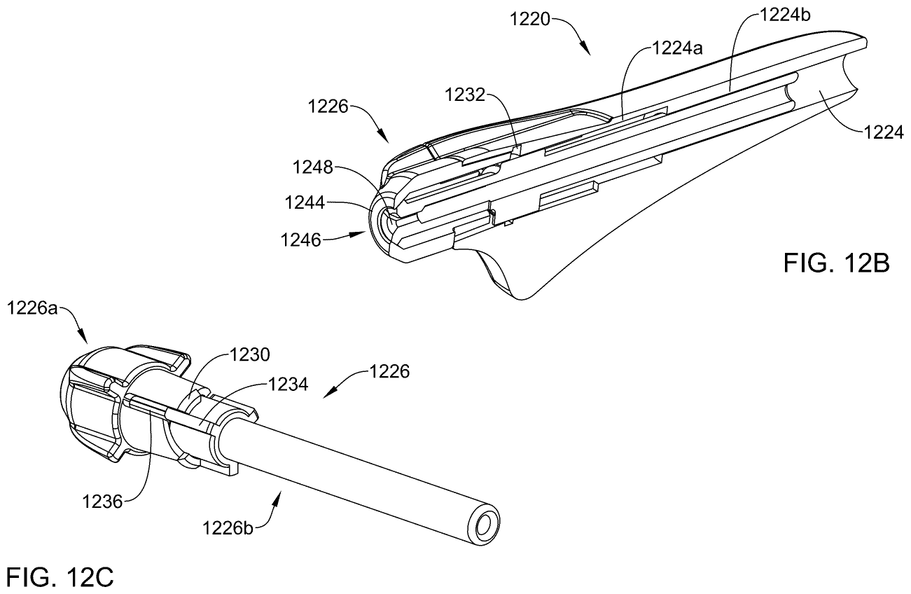

FIG. 12A is a perspective view of an illustrative assembly 1220, shown in a closed configuration and FIG. 12B is a cross-sectional view taken along the line 12B-12B of FIG. 12A. The illustrative assembly 1220 may be considered as being a trigger twist embodiment. The assembly 1220 includes an assembly body 1222 that defines a channel 1224 extending through the assembly body 1222. The channel 1224 allows the puller 101/201 to pass through the assembly 1220. The channel 1224 also accommodates an engagement feature 1226 that is partially disposed distal of the assembly body 1222 and is partially disposed within the channel 1224. In some instances, the channel 1224 includes a distal section 1224a and a proximal section 1224b. The distal section 1224a may have a larger diameter than the proximal section 1224b, and may be adapted to accommodate a larger diameter portion of the engagement feature 1226 while the proximal section 1224b may be adapted to accommodate a smaller diameter portion of the engagement feature 1226.

The engagement feature 1226 may be movable between an open configuration in which the engagement member 316 is free to move relative to the engagement feature 1226 and a closed configuration in which the engagement member 316 is captured by the engagement feature 1226. The engagement feature 1226 includes a first engagement feature element 1126a and a second engagement feature element 1126b. FIG. 12C provides a perspective view of the engagement feature 1226, showing the interaction between the first engagement feature element 1126a and the second engagement feature element 1126b. In FIG. 12C, the engagement feature 1226 is shown in an open configuration.

The first engagement feature element 1126a includes an annular ring 1230 that is adapted to engage a corresponding annular recess 1232 formed within the assembly body 1222 proximate the channel 1224. The annular ring 1230 may form a snap-fit with the annular recess 1232 in order to secure the first engagement feature element 1126a relative to the assembly body 1222 while allowing the first engagement feature element 1126a to rotate relative to the second engagement feature element 1126b.

As can be seen in FIG. 12C, the second engagement feature element 1126b includes one or more axially extending protrusions 1234 that engage one or more corresponding axially extending slots within the first engagement feature element 1126a. Rotating the first engagement feature element 1126a relative to second engagement feature element 1126b causes the second engagement feature element 1226 to temporarily back away from the first engagement feature element 1126a. The second engagement feature element 1126b includes a distal end 1246 (as designated in FIGS. 12A and 12B) having an enlarged diameter, and is axially divided into several segments such as that shown in FIG. 11D.

It will be appreciated that in the closed configuration, a distal opening 1244 (as designated in FIG. 12B) of the first engagement feature element 1126a will hold the segments together, defining a small aperture 1248 (as designated in FIGS. 12A and 12B) therebetween that is dimensioned to allow the puller 101/201 to pass through, but will not allow the engagement member 316 to pass through. Axial movement between the first engagement feature element 1126a and the second engagement member element 1126b, the segments are free to move radially outwardly, temporarily increasing the diameter of the aperture 1248 to a point that the engagement member 316 is able to pass through the aperture 1248 and reach a larger diameter recess within the second engagement feature element 1126b that is dimensioned to accommodate the engagement member 316. Once the engagement member 316 is disposed within the recess within the second engagement feature element 1126b, a user is able to exert a force on the puller 101/201 by pulling the assembly 1220 in a proximal direction.

In some instances, the assembly body 1222 may include one or more features that render the assembly body 1222 more ergonomic. As an example, the assembly body 1222 may include a trigger 1250 that allows a user to pull proximally on the assembly body 1222. In some instances, the more proximal portions of the assembly body 1222 may also be shaped to more naturally accommodate the user's hand as the user wraps their hand around the assembly body 1222.

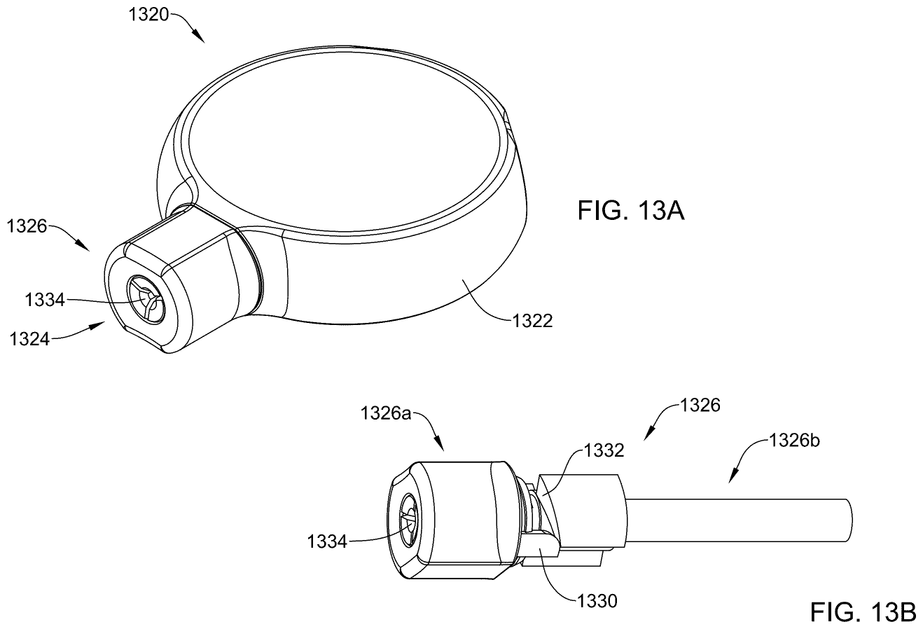

FIG. 13A is a perspective view of an illustrative assembly 1320, shown in a closed configuration. The illustrative assembly 1320 may be considered as being a twist embodiment. The assembly 1320 includes an assembly body 1322 that defines a channel 1324 extending through the assembly body 1322. The channel 1324 allows the puller 101/201 to pass through the assembly 1320. The channel 1324 also accommodates an engagement feature 1326. The engagement feature 1326 may be movable between an open configuration in which the engagement member 316 is free to move relative to the engagement feature 1326 and a closed configuration in which the engagement member 316 is captured by the engagement feature 1326. FIG. 13B is a perspective view of the engagement feature 1326, showing a first engagement feature element 1326a and a second engagement feature element 1326b.

The first engagement feature element 1326a includes a cam bar 1330 that engages a corresponding cam surface 1332 defined on the second engagement feature element 1326b. Other forms of converting relative rotation between the first engagement feature element 1326a and the second engagement feature element 1326b into relative translation between the first engagement feature element 1326a and the second engagement feature element 1326b are also contemplated.

It will be appreciated that relative rotation between the first engagement feature element 1326a and the second engagement feature element 1326b will cause the cam bar 1330 to move against the cam surface 1332, thereby causing relative translation between the first engagement feature element 1326a and the second engagement feature element 1326b. This will allow a distal end 1334 of the second engagement feature element 1326b to expand enough to allow the engagement member 316 to pass through into a larger recess formed within the second engagement feature element 1326b, meaning that the engagement feature 1326 is temporarily in the open configuration. Relative rotation between the first engagement feature element 1326a and the second engagement feature element 1326b in an opposing direction will cause translation in the opposing direction, returning the engagement feature 1326 to the closed configuration.

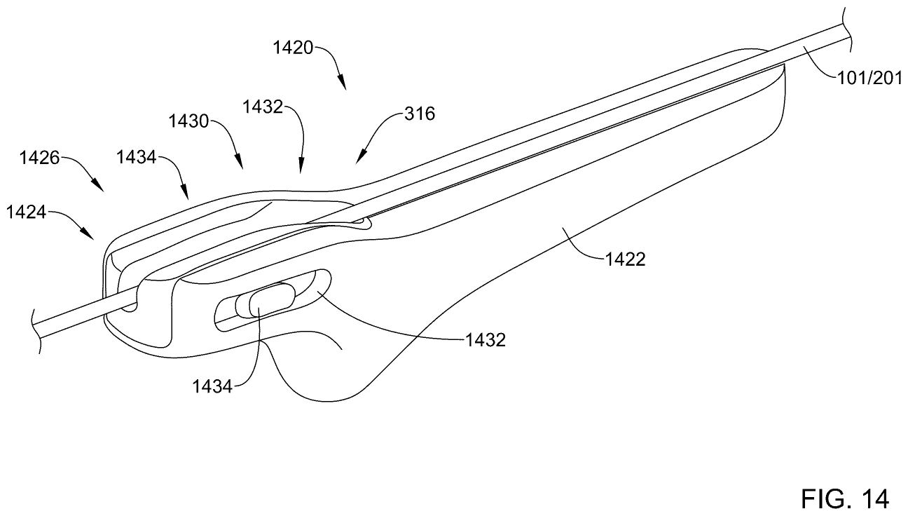

In some instances, an assembly may be adapted to provide an indication of how much force is being applied. In some instances, having to pull harder than a particular force may be an indication that there is something possibly wrong, and rather than simply pulling even harder, it may be beneficial to stop and investigate. FIGS. 14 through 17B provide examples of assemblies that are adapted to provide an indication of applied force.

FIG. 14 is a perspective view of an illustrative assembly 1420, shown in combination with the puller 101/201. While not visible, the puller 101/201 includes the engagement member 316. The assembly 1420 includes an assembly body 1422. The assembly body 1422 defines an engagement feature void 1430. An engagement feature 1426 is disposed within the engagement feature void 1430. The assembly body 1422 also defines a pair of slots 1432 on either side of the assembly body 1422 that are coupled with the engagement feature void 1430, although only one slot 1432 is visible. The engagement feature 1426 is adapted to translate within the engagement feature void 1430 in response to applied force.

The engagement feature 1426 defines a channel 1424 extending through the engagement feature 1426, including a first channel section 1424a having a smaller diameter adapted to accommodate the puller 101/201 and a second channel section 1426b having a larger diameter adapted to accommodate the engagement member 316. Pulling proximally on the assembly body 1422 with the engagement member 316 disposed within the second channel section 1426b will cause the puller 101/201 to be pulled proximally. The engagement feature 1426 includes two protrusions 1434 on either side of the engagement feature 1426 that extend through the corresponding slots 1432. The position of each protrusion 1434 relative to each slot 1432 will vary, depending on the applied force.

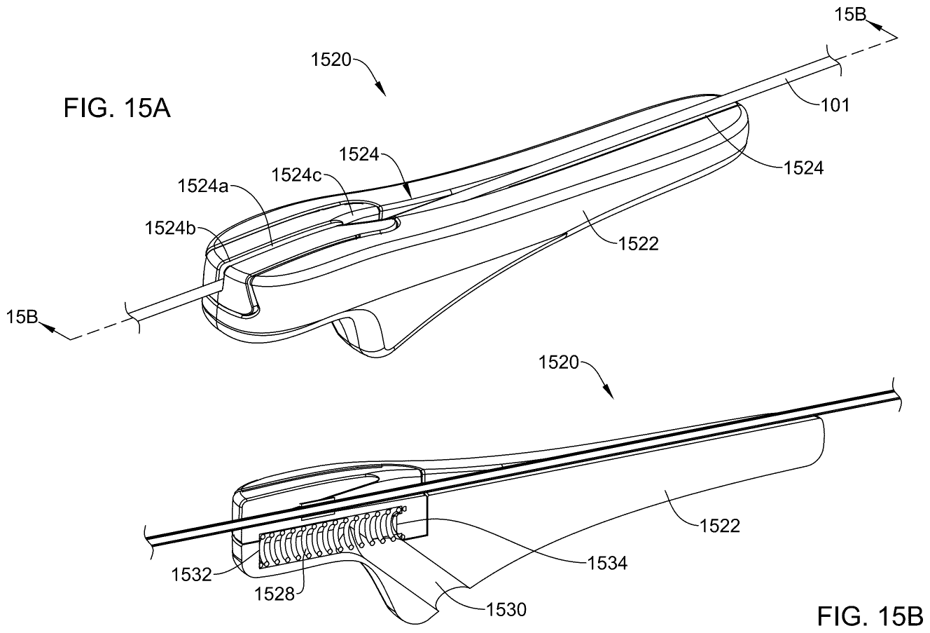

The engagement feature 1426 may be adapted to have a particular resistance against movement relative to the engagement feature void 1430. In some instances, an internal spring may instead be used to provide the engagement feature 1426 with resistance to movement. When the applied force exceeds this resistance, the engagement feature 1426 will move relative to the engagement feature void 1430. The position of the protrusions 1432 relative to the slots 1432 will provide an indication that perhaps too much force is being applied. In some instances, parts of the engagement feature 1426 may be color coded, so that the color visible through the slots 1432 provides an indication of the relative applied force. For example, parts of the engagement feature 1426 may be green, yellow and red, with green indicating a safe force, yellow indicating an applied force that is getting close to being excessive, and red indicating an applied force that is excessive. While not shown, the assembly 1420 may include an internal spring (similar to the spring 1528 shown in FIG. 15B) to bias the engagement feature 1426.

FIG. 15A is a perspective view of an illustrative assembly 1520, shown in combination with the puller 101/201 and FIG. 15B is a cross-sectional view taken along the line 15B-15B of FIG. 15A. While not visible, the puller 101/201 includes the engagement member 316. The assembly 1520 includes an assembly body 1522. The assembly body 1522 defines a channel 1524 that extends into an engagement feature void 1530. An engagement feature 1526 is disposed within the engagement feature void 1530.

The engagement feature 1526 defines a channel 1524a extending through the engagement feature 1526. The channel 1524a functions as a continuation of the channel 1524 extending through the assembly body 1522, and includes a first channel section 1524b having a smaller diameter adapted to accommodate the puller 101/201 and a second channel section 1526c having a larger diameter adapted to accommodate the engagement member 316. Pulling proximally on the assembly body 1522 with the engagement member 316 disposed within the second channel section 1526c will cause the puller 101/201 to be pulled proximally.

A spring 1528 may be advanced through a passageway 1530 that passes through the assembly body 1522 to a position where the spring 1528 extends between a first bearing surface 1532 formed within the assembly body 1522 and a second bearing surface 1534 formed within the engagement feature 1526. As force is applied to the engagement feature 1526 via the engagement member 316, the engagement feature 1526 will remain stationary until the applied force starts to exceed the biasing force applied by the spring 1528. As the spring 1528 begins to compress, the engagement feature 1526 will start to move relative to the assembly body 1522. This movement provides an indication that the applied force may be getting excessive. Various springs with differing spring constant may be used as the spring 1528. For example, a spring with a lower spring constant may be used if a lower pulling force is desired, and a spring with a higher spring constant may be used if a higher pulling force is desired.

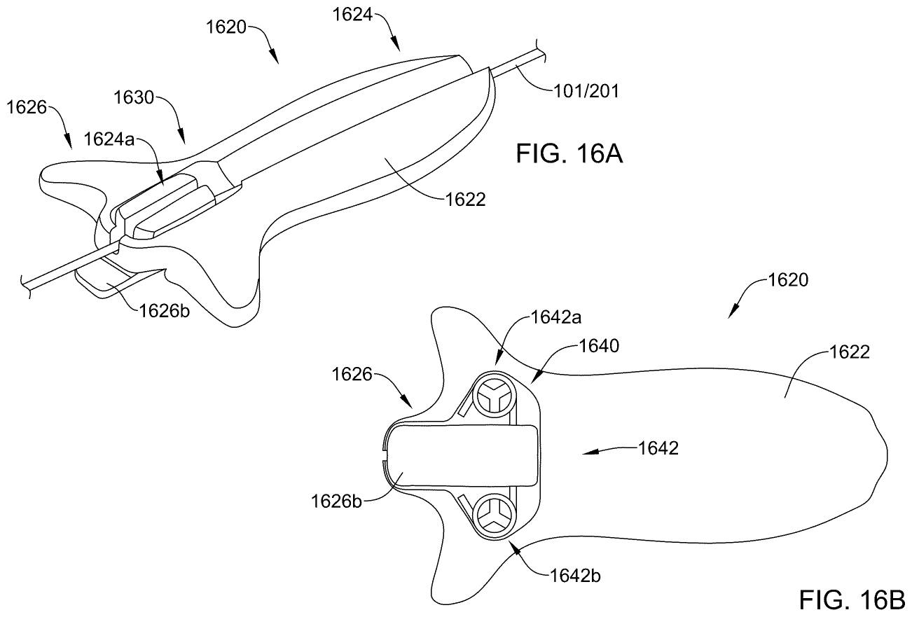

FIG. 16A is a perspective view and FIG. 16B is a bottom plan view of an illustrative assembly 1620. While not visible, the puller 101/201 includes the engagement member 316. The assembly 1620 includes an assembly body 1622. The assembly body 1622 defines a channel 1624 that extends into an engagement feature void 1630. An engagement feature 1626 is disposed within the engagement feature void 1630. The engagement feature 1626 includes a first engagement feature element 1626a that includes a channel 1624a that provides a continuation for the channel 1624 such that the puller 101/201 may extend all the way through the assembly 1620. The engagement feature 1626 also includes a second engagement feature element 1626b that moves with the first engagement feature element 1626a.

In some instances, the engagement feature void 1630 is longer than the first engagement feature element 1626a, meaning that the first engagement feature element 1626a is able to translate within the engagement feature void 1630. The underside of the assembly body 1622 includes a spring assembly void 1640 that accommodates a torsion spring assembly 1642. The torsion spring assembly 1642 includes a first torsion spring 1642a that engages a wall of the spring assembly void 1640 and extends into the engagement feature 1626 and a second torsion spring 1642b that engages a wall of the spring assembly void 1640 and extend into the engagement feature 1626. As force is applied by virtue of the engagement member 316 being captured within the first engagement feature element 1626a, the engagement feature 1626 will begin to translate relative to the assembly body 1622 as the applied force exceeds the biasing force applied by the torsion spring assembly 1640. This movement provides an indication that the applied force may be getting excessive. Various torsion springs with differing spring rates may be used in the torsion spring assembly 1640.

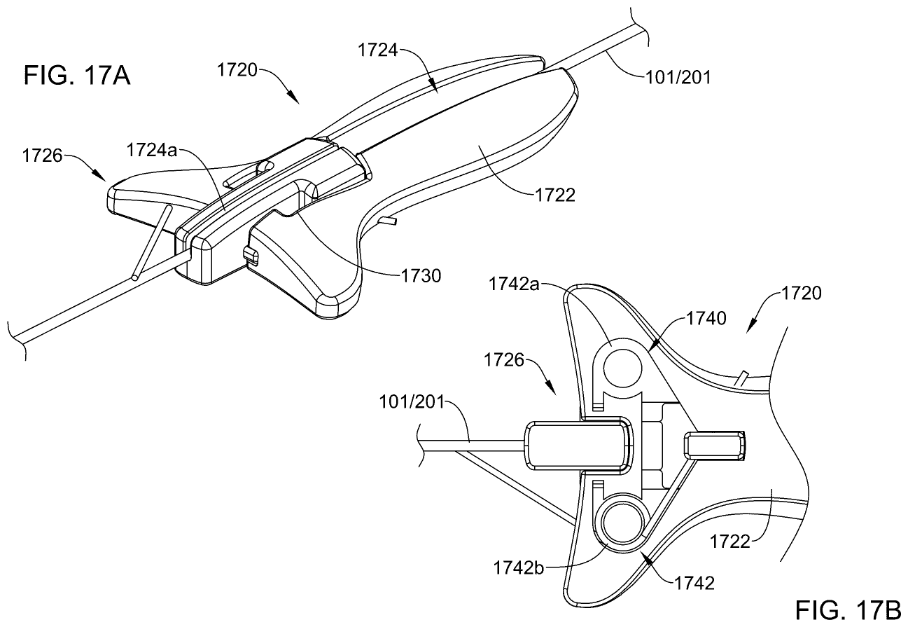

FIG. 17A is a perspective view and FIG. 17B is a bottom plan view of an illustrative assembly 1720. While not visible, the puller 101/201 includes the engagement member 316. The assembly 1720 includes an assembly body 1722. The assembly body 1722 defines a channel 1724 that extends into an engagement feature void 1730. An engagement feature 1726 is disposed within the engagement feature void 1730. The engagement feature 1726 includes a channel 1724a that provides a continuation for the channel 1724 such that the puller 101/201 may extend all the way through the assembly 1720.

In some instances, the engagement feature void 1730 is longer than the engagement feature element 1722, meaning that the engagement feature element 1726 is able to translate within the engagement feature void 1730. The underside of the assembly body 1722 includes a spring assembly void 1740 that accommodates a torsion spring assembly 1742. The torsion spring assembly 1742 includes a first torsion spring 1742a and a second torsion spring 1742b. As force is applied by virtue of the engagement member 316 being captured within the engagement feature 1726, the engagement feature 1726 will begin to translate relative to the assembly body 1722 as the applied force exceeds the biasing force applied by the torsion spring assembly 1740. This movement provides an indication that the applied force may be getting excessive. Various torsion springs with differing spring rates may be used in the torsion spring assembly 1740.

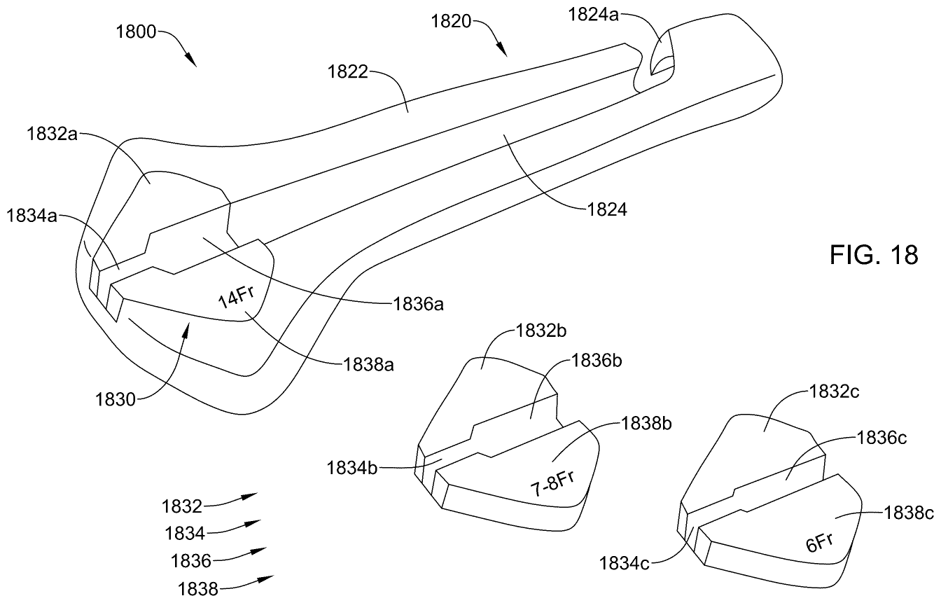

FIG. 18 is a perspective view of an illustrative assembly kit 1800 that is customizable for differing puller sizes. The assembly kit 1800 includes a assembly 1820 having an assembly body 1822. The assembly body 1822 defines a channel 1824 extending through the assembly body 1822. In some instances, the channel 1824 includes a hook portion 1824a into which the puller 101/201 may be hooked in order to keep the puller 101/201 coupled to the assembly 1820. An insert void 1830 is formed within the assembly body 1822 and is coupled with the channel 1824.

The insert void 1830 is adapted to accommodate any of a variety of different inserts 1832, individually labeled as 1832a, 1832b and 1832c. The assembly kit 1800 may include any number of inserts 1832. Each of the inserts 1832 includes a first channel 1834, individually labeled as 1834a, 1834b and 1834c, that is dimensioned to accommodate the puller 101/201 and a second channel 1836, individually labeled as 1836a, 1836b and 1836c, that is dimensioned to accommodate the engagement member 316. In some instances, each insert 1832 may be a different color, to make it easier to identify which insert 1832 is appropriate. In some instances, each insert 1832 may include an insignia 1838, individually labeled as 1838a, 1838b and 1838c, that provides a numerical or other indication of the size puller 101/201 that the particular insert 1832 is intended for.

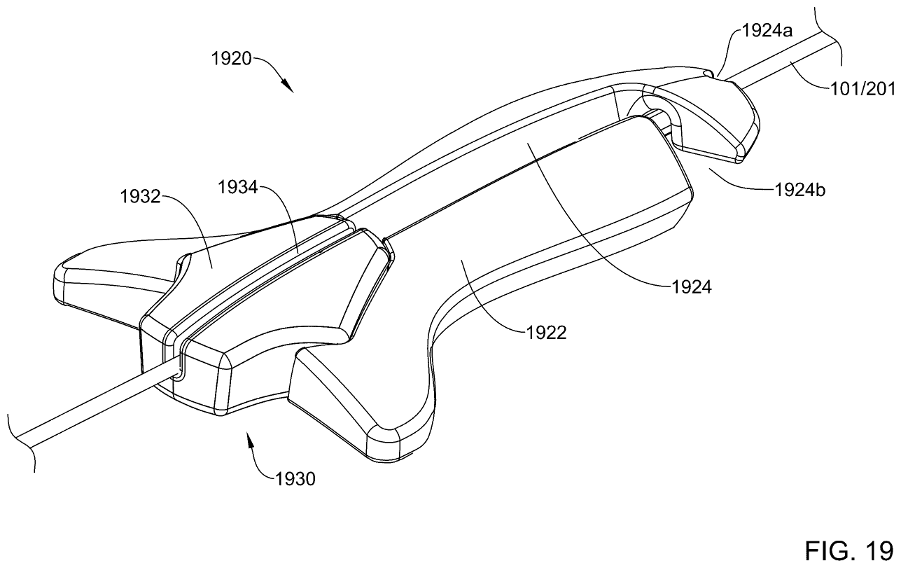

FIG. 19 is a perspective view of an illustrative assembly 1900 including an insert that is customizable for differing puller sizes. The assembly 1920 has an assembly body 1922. The assembly body 1922 defines a channel 1924 extending through the assembly body 1922. An insert void 1930 is formed within the assembly body 1922 and is coupled with the channel 1924. The insert void 1930 is adapted to accommodate an insert 1932, which may be available for any of a variety of different sized pullers 101/201. In some instances, the channel 1924 may be axially aligned with a channel extension 1924a that is adapted to allow the puller 101/201 to pass through the channel extension 1924a. In some instances, the channel 1924 may also include a hook portion 1924b that allows the puller 101/201 to be advanced into the channel 1924.

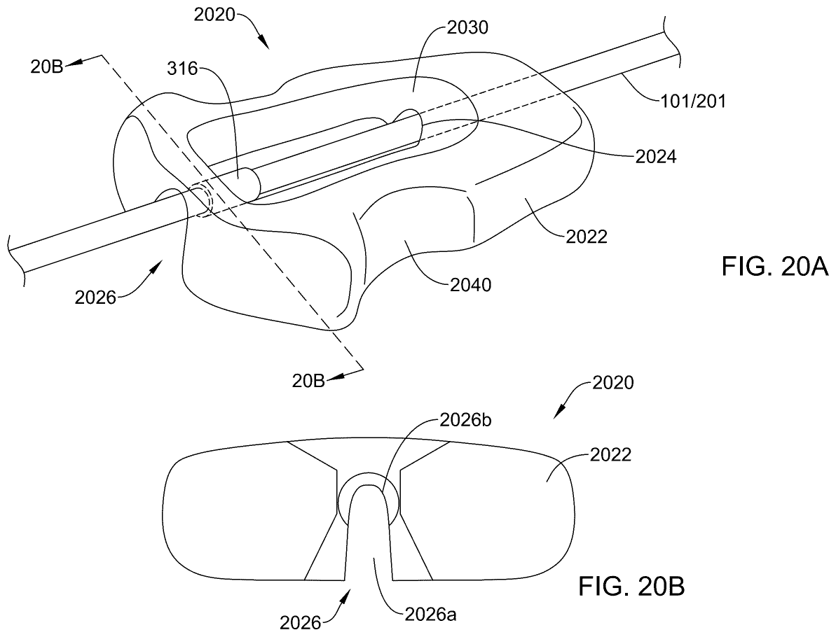

FIG. 20A is a perspective view of an illustrative assembly 2020 and FIG. 20B is a cross-sectional view thereof taken along the line 20B-20B of FIG. 20A. The assembly 2020 includes an assembly body 2022. The assembly body 2022 defines a window 2030 that extends through the assembly body 2022 from a front side to a back side. The window 2030 provides a visual clue that the engagement member 316 is locked in place. The window 2030 also permits a user to grip the assembly 2020 and the puller 101/201 simultaneously. In some instances, the assembly body 2022 also defines a channel 2024 that extends from the window 2030 and allows the puller 101/201 to extend through the channel 2024. The assembly 2020 includes an engagement feature 2026 having a channel 2026a that is dimensioned to accommodate both the puller 101/201 and the engagement member 316. The channel 2026a extends to an engagement surface 2026b that is larger in diameter than the puller 101/201 but is smaller in diameter than the engagement member 316 so that the assembly 2020 may be used to pull the puller 101/201 by engaging the engagement member 316. The assembly body 2022 may also include finger grips 2040.

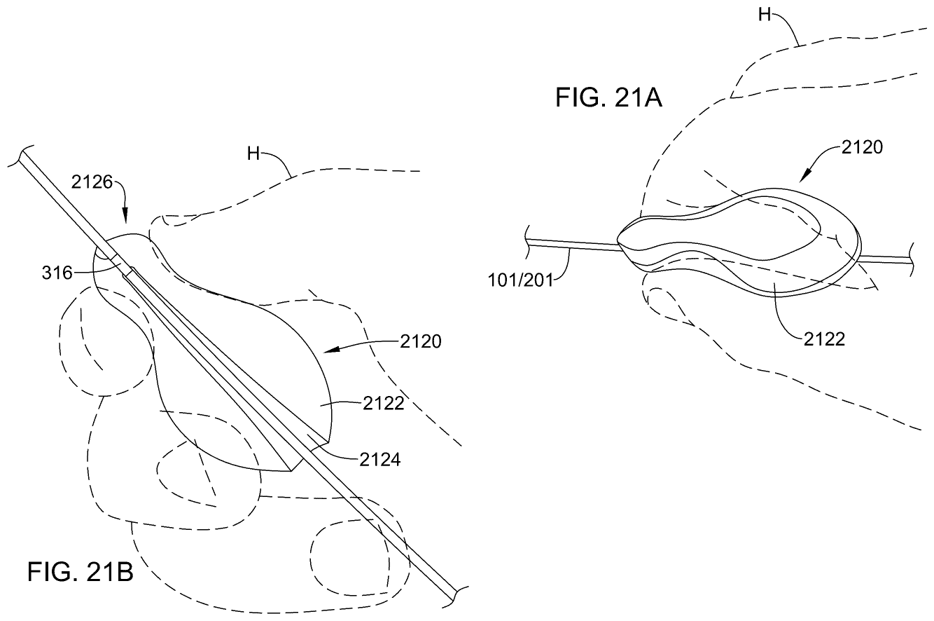

FIG. 21A is a perspective view of an illustrative assembly 2120, shown in combination with a puller 101/201 in the grip of a user's hand H while FIG. 21B is a rear perspective view of the illustrative assembly 2120, shown in combination with a puller 101/201 in the grip of a user's hand H. The assembly 2120 includes an assembly body 2122 that may be considered as representing the shape of a traditional computer mouse, having a widened portion and a narrowed portion to better fit the hand H. As seen in FIG. 21B, the back of the assembly body 2122 includes a channel 2124 that accommodates the puller 101/201 (and attached engagement member 316) therein. The assembly 2120 includes an engagement feature 2126 that is adapted to releasably engage the engagement member 316 and that may be similar to the engagement feature 2026 shown in FIG. 20B. The user is holding the assembly 2120 in their hand H, with the assembly body 2122 held between the user's thumb and index finger.

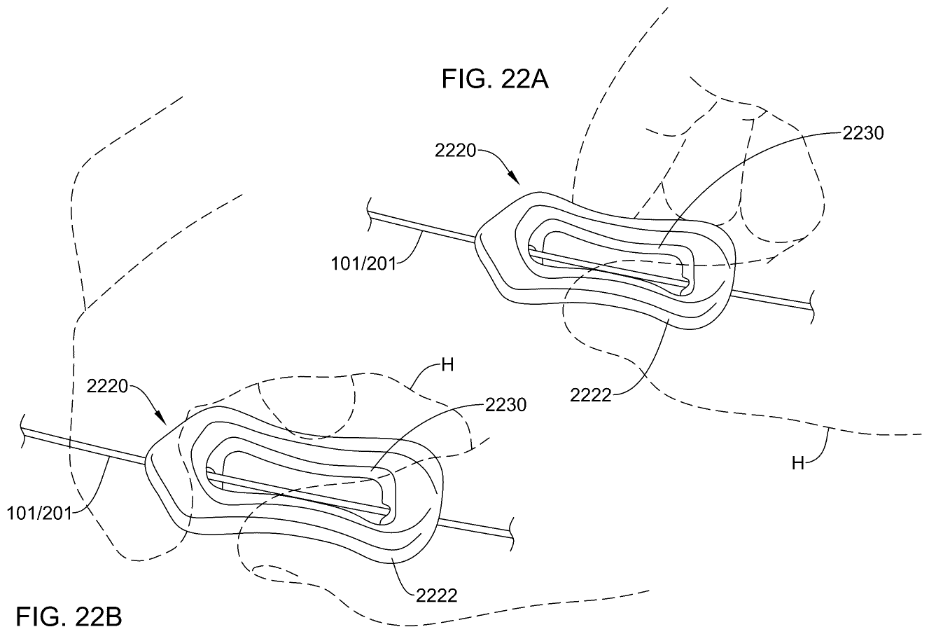

FIG. 22A is a perspective view of an illustrative assembly 2220, shown in combination with a puller 101/201 in the grip of a user's hand H in a first orientation while FIG. 22B is a perspective view of the illustrative assembly 2120, shown in combination with a puller 101/201 in the grip of a user's hand H in a second orientation. The assembly 2220 includes an assembly body 2222 defining a window 2230. In comparing FIG. 22A with FIG. 22B, it can be seen that the assembly 2220 may be held in a user's hand H in several different orientations.

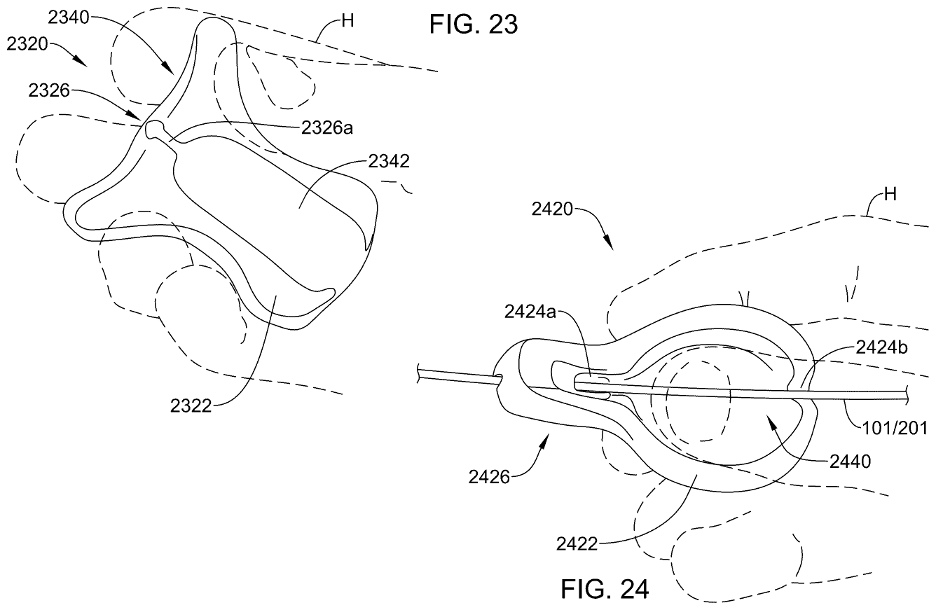

FIG. 23 is a perspective view of an illustrative assembly 2320, shown in the grip of a user's hand H. The assembly 2320 includes an assembly body 2322. The assembly body 2322 defines a crossbow shape 2340 and an engagement feature 2326 disposed near the crossbow shape 2340. The crossbow shape 2340 enables grasping and pulling the assembly body 2322. The engagement feature 2326 includes a channel 2326a that is dimensioned to accommodate the puller 101/201 but not to allow the engagement member 316 to pass. A void 2342 is formed along an underside of the assembly body 2322 and facilitates grasping of the assembly body 2322. The void 2342 may also accommodate the engagement member 316.

FIG. 24 is a perspective view of an illustrative assembly 2420, shown in the grip of a user's hand H. The assembly 2420 includes an assembly body 2422. The assembly body 2422 defines a window 2440 and a channel 2424 including a first channel portion 2424a near an engagement feature 2426 and a second channel portion 2424b on an opposite side of the window 2440. The engagement feature 2326 is adapted to allow the puller 101/201 to pass through but not allow the engagement member 216 to pass through. The shape of the assembly body 2422, including the window 2440, may facilitate grasping of the assembly body 2422.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. An assembly for engaging a medical device shaft, comprising:
an assembly body defining a channel adapted to accommodate the medical device shaft extending therethrough, the assembly body adapted to be held in a person's hand;
an engagement feature on the assembly body adapted to selectively engage an engagement member secured on the medical device shaft;
wherein the engagement feature is movable between an open configuration in which the engagement feature does not engage the engagement member and a closed configuration in which the engagement feature engages the engagement member.

2. The assembly of claim 1, wherein the engagement feature comprises:
a first engagement feature element molded into the assembly body, the first engagement feature element biased into the open configuration; and
a second engagement feature slidingly disposed relative to the first engagement feature such that sliding the second engagement feature over the first engagement feature element moves the first engagement feature element into the closed configuration.

3. The assembly of claim 1, further comprising a lever pivotably secured to the assembly body, the engagement feature comprising a tab extending towards the channel, the tab extending into the channel when the engagement feature is in the closed configuration.

4. The assembly of claim 1, wherein:
the assembly body comprises:
a first assembly body half extending from a assembly body midpoint;
a second assembly body half extending from the assembly body midpoint;
the engagement feature comprises:
a first engagement feature element disposed within the first assembly body half; and
a second engagement feature element disposed within the second assembly body half;
wherein the first assembly body half and the second assembly body half are biased to a configuration in which the first engagement feature element and the second engagement feature element are spaced apart in the open configuration; and
wherein the first assembly body half and the second assembly body half are adapted to be squeezed together in order to move the first engagement feature element and the second engagement feature element together into the closed configuration.

5. The assembly of claim 1, wherein the engagement feature comprises a spring-loaded collet disposed within the channel that is adapted to push against one or more ramp surfaces within the assembly body;
wherein the spring-loaded collet opens to accommodate the engagement member as the engagement member passes, then closes to hold engagement member.

6. The assembly of claim 5, wherein the engagement feature further comprises a twist element that when rotated causes the spring-loaded collet to open.

7. The assembly of claim 5, further comprising sliders disposed on either side of the assembly body that are operably coupled with the spring-loaded collet, where the sliders are adapted to allow manually opening the spring-loaded collet by sliding the sliders.

8. The assembly of claim 1, wherein the assembly body comprises:
a first assembly body section;
a second assembly body section hingedly coupled to the first assembly body section;
the engagement feature includes a first engagement feature element disposed within the first assembly body section and a second engagement feature element disposed within the second assembly body section;
wherein the second housing section is adapted to pivot away from the first assembly body section in the open configuration and to pivot towards the first assembly body section in the closed configuration.

9. The assembly of claim 8, wherein the second assembly body section is hingedly coupled to the first assembly body section via a living hinge.

10. The assembly of claim 8, wherein the first engagement feature element and the second engagement feature element are each disposed at an end of the assembly body opposite that of the living hinge.

11. The assembly of claim 9, wherein the first engagement feature element and the second engagement feature element are each disposed proximate the living hinge.

12. The assembly of claim 1, wherein the engagement feature comprises:
a first engagement feature element including a first jaw portion and a first handle portion; and
a second engagement feature element including a second jaw portion and a second handle portion;
the first jaw portion biased into contact with the second jaw portion.

13. The assembly of claim 12, wherein:
the first jaw portion includes a first ramp portion;
the second jaw portion includes a second ramp portion; and
the first ramp portion and the second ramp portion together allow the engagement member to urge the first jaw portion and the second jaw portion away from their biased position such that the engagement member can be pushed into engagement with the engagement feature.

14. The assembly of claim 12, wherein:
the assembly body includes a first side aperture and a second side aperture;
the first handle portion extends out the first side aperture;
the second handle portion extends out the second side aperture; and
the first handle portion and the second handle portion are adapted to be pushed in order to separate the first jaw portion and the second jaw portion.

15. The assembly of claim 1, further comprising a force gauge.

16. The assembly of claim 1, wherein the assembly body includes an ergonomic shape to facilitate being held in a person's hand.

17. A assembly for engaging a medical device shaft, comprising:
an assembly body defining a channel adapted to accommodate the medical device shaft extending therethrough, the assembly body adapted to be held in a person's hand;
an engagement feature adapted to engage an engagement member secured on the medical device shaft; and
a force gauge secured relative to the assembly body, the force gauge adapted to provide an indication of an applied force.

18. The assembly of claim 17, wherein the assembly body includes an ergonomic shape to facilitate being held in a person's hand.

19. The assembly of claim 17, wherein the engagement feature is adapted to releasably engage the engagement member.

20. A assembly for engaging a medical device shaft, the medical device shaft including an engagement member, the assembly comprising:
an ergonomic assembly body defining a channel adapted to accommodate the medical device shaft extending therethrough, the ergonomic assembly body adapted to be held in a person's hand; and
an engagement feature adapted to selectively engage an engagement member secured on the medical device shaft, where the engagement feature is movable between an open configuration in which the engagement feature does not engage the engagement member and a closed configuration in which the engagement feature engages the engagement member.
